(19) United States
(12) Patent Application Publication
(10) Pub. No.: US 2013/0053816 A1
(43) Pub. Date: Feb. 28, 2013

(54) MULTI-RESERVOIR INFUSION PUMP SYSTEMS AND METHODS

(71) Applicants: Paul M. DiPERNA, Cardiff, CA (US); Steve GRIFFIE, Santa Ana, CA (US)

(72) Inventors: Paul M. DiPERNA, Cardiff, CA (US); Steve GRIFFIE, Santa Ana, CA (US)

(73) Assignee: TANDEM DIABETES CARE, INC., San Diego, CA (US)

(21) Appl. No.: 13/557,163

(22) Filed: Jul. 24, 2012

Related U.S. Application Data
(60) Provisional application No. 61/511,220, filed on Jul. 25, 2011.

(51) Int. Cl. A61M 5/142; F16J 9/28

(52) U.S. Cl. 604/500; 604/151; 277/434

(57) ABSTRACT

Embodiments are directed to multi-reservoir infusion devices, systems, and methods of using the same for dispensing materials. In some cases, the devices, systems and methods may be used for infusing a material such as medicament, e.g., insulin, into a body in need thereof.

### RELATED APPLICATIONS

This application claims priority under 35 U.S.C. 119(e) from U.S. Provisional Patent Application No. 61/511,220, filed Jul. 25, 2011, by Paul M. DiPerna and Steve Griffie, entitled MULTI-RESERVOIR INFUSION PUMP SYSTEMS AND METHODS, designated by Attorney Docket No. TDM-1510-PV, which is incorporated by reference herein in its entirety.

This application is also related to U.S. patent application Ser. No. 12/846,688, Attorney Docket No. TDM-1500-UT, entitled INFUSION PUMP SYSTEM WITH DISPOSABLE CARTRIDGE HAVING PRESSURE VENTING AND PRESSURE FEEDBACK, filed Jul. 29, 2010, by P. DiPerna, et al., U.S. patent application Ser. No. 12/846,706, Attorney Docket No. TDM-1500-UT2, entitled INFUSION PUMP SYSTEM WITH DISPOSABLE CARTRIDGE HAVING PRESSURE VENTING AND PRESSURE FEEDBACK, filed Jul. 29, 2010, by M. Michaud, et al., U.S. patent application Ser. No. 12/846,720, Attorney Docket No. TDM-1500-UT3, entitled INFUSION PUMP SYSTEM WITH DISPOSABLE CARTRIDGE HAVING PRESSURE VENTING AND PRESSURE FEEDBACK, filed Jul. 29, 2010, by P. DiPerna, et al., U.S. patent application Ser. No. 12/846,733, Attorney Docket No. TDM-1500-UT4, entitled INFUSION PUMP SYSTEM WITH DISPOSABLE CARTRIDGE HAVING PRESSURE VENTING AND PRESSURE FEEDBACK, filed Jul. 29, 2010, by M. Michaud, et al., and U.S. patent application Ser. No. 12/846,734, Attorney Docket No. TDM-1500-UT5, entitled INFUSION PUMP SYSTEM WITH DISPOSABLE CARTRIDGE HAVING PRESSURE VENTING AND PRESSURE FEEDBACK, filed Jul. 29, 2010, by E. Verhoef, et al., International (PCT) Patent Application No. PCT/US2010/043789, Attorney Docket No. TDM-1500-PC, entitled INFUSION PUMP SYSTEM WITH DISPOSABLE CARTRIDGE HAVING PRESSURE VENTING AND PRESSURE FEEDBACK, filed Jul. 29, 2010, by D. Brown et al; U.S. patent application Ser. No. 12/714,299, entitled METHODS AND DEVICES FOR DETERMINATION OF FLOW RESERVOIR VOLUME, filed Feb. 26, 2010 by M. Rosinko et al.; U.S. Pat. No. 7,008,403, entitled INFUSION PUMP AND METHOD FOR USE by S. Mallett; U.S. Pat. No. 7,341,581, entitled INFUSION PUMP AND METHOD FOR USE by S. Mallet; U.S. Pat. No. 7,374,556, entitled INFUSION PUMP AND METHOD FOR USE, by S. Mallet; U.S. Patent Application Publication No. 2007/0264130, entitled INFUSION PUMPS AND METHOD FOR USE, filed May 4, 2007 by S. Mallett; and U.S. Patent Application Publication No. 2009/0191067, entitled TWO CHAMBER PUMPS AND RELATED METHODS, filed Jan. 25, 2008 by P. DiPerna, all of which are incorporated by reference herein in their entirety.

### FIELD OF THE INVENTION

Embodiments may be directed to infusion devices or systems and methods of using these devices or systems for dispensing fluids in a controllable and reliable manner. In some cases, embodiments may include portable infusion pumps and methods of using such pumps for infusing a fluid material or multiple fluid materials, such as liquid medicaments (including drugs or therapeutic agents), to a patient or any other suitable destination. Some liquid medicaments suitable for delivery to a patient by device and method embodiments discussed herein may include insulin, antibiotics, glucose, saline or any other suitable liquid medicament.

### BACKGROUND

There are many applications in academic, industrial, and medical fields, as well as others, that may benefit from devices and methods that are capable of accurately and controllably delivering fluids, including liquids and gases that have a beneficial effect when administered in known and controlled quantities. Such devices and methods may be particularly useful in the medical field where much of the treatment for a large percentage of patients includes the administration of a known amount of a substance at predetermined intervals. In some instances, it may be desirable to safely, reliably and comfortably administer required doses of more than one medication or other fluid to a patient from a single device.

What have been needed are devices and methods capable of reliably delivering two or more therapeutic substances from a single device in an efficient manner. Such devices and methods may be useful in some cases in order to facilitate patient compliance, accurate treatment of medical conditions requiring multiple medicaments as well as other applications or indications.

### SUMMARY

Some embodiments of an infusion pump cartridge for delivering fluid to a patient, include a delivery mechanism having a first inlet port, a second inlet port spaced from the first inlet port, and at least one outlet port which is spaced from the first inlet port and second inlet port. The delivery mechanism may also include at least one constrained variable volume that is translatable between a position in fluid communication with the first inlet port, a position in fluid communication with the second inlet port and a position in fluid communication with the outlet port and which is configured to expand while in fluid communication with an inlet port and contract while in fluid communication with an outlet port. The cartridge may also include a first fluid reservoir including an interior volume in fluid communication with the first inlet port and a second fluid reservoir including an interior volume in fluid communication with the second inlet port. For some such embodiments, the constrained variable volume may be configured to expand and contract due to exertion of a translational force on a boundary section of the constrained variable volume.

Some embodiments of an infusion pump for delivering fluids to a patient may include a delivery mechanism having a first inlet port, a second inlet port spaced from the first inlet port, and at least one outlet port which is spaced from the first inlet port and second inlet port. The delivery mechanism may also include at least one constrained variable volume translatable between a position in fluid communication with the first inlet port, a position in fluid communication with the second inlet port and a position in fluid communication with the outlet port. The infusion pump may further have a first fluid reservoir including an interior volume in fluid communication with the first inlet port, a second fluid reservoir including an interior volume in fluid communication with the second inlet port, and a drive mechanism. The drive mechanism may be operatively coupled to the constrained variable volume and be configured to translate the constrained variable volume from each of the inlet ports to an outlet port. The drive mechanism may also be configured to expand and contract the constrained variable volume of the spool. For some embodiments such as this, the drive mechanism may be configured to expand or contract the constrained variable volume by exerting translational force through a boundary section of the constrained variable volume.

Some embodiments of an infusion pump for delivering fluids to a patient may include a delivery mechanism having an axial bore with a longitudinal axis and an interior volume, a first inlet port in fluid communication with the interior volume of the axial bore, and a second inlet port axially spaced from the first inlet port and in fluid communication with the interior volume of the axial bore. The delivery mechanism may also include at least one outlet port which is axially spaced from the first inlet port and a second inlet port and which is in fluid communication with the interior volume of the axial bore. A spool may be disposed within the axial bore and be axially translatable within the axial bore. The spool may also form a constrained variable volume in conjunction with an interior surface of the axial bore. The infusion pump may also include a first fluid reservoir including an interior volume in fluid communication with the first inlet port and a second fluid reservoir including an interior volume in fluid communication with the second inlet port. A drive mechanism may be operatively coupled to the spool and be configured to axially translate the constrained variable volume from each of the inlet ports to an outlet port and configured to expand or contract the constrained variable volume of the spool by exerting translational axial force through a boundary section of the constrained variable volume. In some cases, the drive mechanism may be configured to expand or contract the constrained variable volume by exerting translational axial force through a boundary section of the constrained variable volume.

Some embodiments of an infusion pump for delivering fluids to a patient may include a disposable component that includes a delivery mechanism. The delivery mechanism may include an axial bore, a first inlet port in fluid communication with an interior volume of the axial bore, a second inlet port axially spaced from the first inlet port and in fluid communication with the interior volume of the axial bore, and at least one outlet port which is axially spaced from the inlet ports and which is in fluid communication with the interior volume of the axial bore. The delivery mechanism may also include a spool which is disposed within the axial bore, that is axially translatable within the axial bore, and which forms a constrained variable volume in conjunction with an interior surface of the axial bore. The disposable component may further have a first fluid reservoir including an interior volume in fluid communication with the first inlet port and a second fluid reservoir including an interior volume in fluid communication with the second inlet port. The infusion pump may further include a reusable component that includes a drive mechanism which is operatively coupled to the spool and configured to impart controlled axial movement on the spool and translate the constrained variable volume from each of the inlet ports to the at least one outlet port and further configured to expand or contract the constrained variable volume of the spool by exerting translational axial force through a boundary section of the constrained variable volume.

Some embodiments of a method of delivering fluid to a patient from a single device including two independent fluid reservoirs include providing an infusion pump that has a delivery mechanism with a first inlet port, a second inlet port spaced from the first inlet port, and at least one outlet port which is spaced from the first inlet port and second inlet port. The delivery mechanism may also have a constrained variable volume translatable between a position in fluid communication with the first inlet port, a position in fluid communication with the second inlet port and a position in fluid communication with the outlet port. The infusion pump may further include a first fluid reservoir including an interior volume in fluid communication with the first inlet port, a second fluid reservoir including an interior volume in fluid communication with the second inlet port and a drive mechanism which is operatively coupled to the constrained variable volume, that is configured to axially translate the constrained variable volume from each of the inlet ports to an outlet port and which is configured to expand or contract the constrained variable volume of the spool. A dispense cycle may be initiated by translating the constrained variable volume into a position in fluid communication with the first inlet port. A translational force may then be exerted through a boundary section of the constrained variable volume to expand the constrained variable volume and draw fluid into the constrained variable through the first inlet port from the first reservoir. The constrained variable volume may then be translated into a position in fluid communication with the outlet port and translational force exerted through a boundary section of the constrained variable volume so as to at least partially contract the constrained variable volume and dispense fluid from the constrained variable volume through the outlet port to a patient. In addition, the constrained variable volume may be translated to a position in fluid communication with the second inlet port and a translational force exerted through a boundary section of the constrained variable volume to expand the constrained variable volume and draw fluid into the constrained variable through the second inlet port from the second reservoir. The constrained variable volume may then be translated to a position in fluid communication with an outlet port and a translational force exerted through a boundary section of the constrained variable volume so as to at least partially contract the constrained variable volume and dispense fluid from the constrained variable volume through the outlet port to a patient. In some such embodiments, the first fluid reservoir may contain a first therapeutic agent for delivery to a patient and the second fluid reservoir may contain a second therapeutic agent different from the first therapeutic agent for delivery to a patient. The method may further include delivering a first therapeutic agent and a second therapeutic agent different from the first therapeutic agent to a patient.

In some cases, such an infusion pump may include at least three inlet ports and respective fluid reservoirs. In such cases, the constrained variable volume may be translated to a position in fluid communication with a third inlet port and a translational force exerted through a boundary section of the constrained variable volume to expand the constrained variable volume and draw fluid into the constrained variable through the third inlet port from a third reservoir. The constrained variable volume may then be translated to a position in fluid communication with an outlet port and a translational force exerted through a boundary section of the constrained variable volume so as to at least partially contract the constrained variable volume and dispense fluid from the constrained variable volume through the outlet port to a patient or any other suitable destination.

Some embodiments of an infusion pump cartridge for delivering fluid to a patient include a delivery mechanism having a first delivery section that includes a first inlet port, a first outlet port, and a first constrained variable volume. The first constrained variable volume which is translatable between a position in fluid communication with the first inlet port and a position in fluid communication with the first outlet port and is configured to expand while in fluid communication with the first inlet port and contract while in fluid communication with the first outlet port. The delivery mechanism may also include a second delivery section that has a second inlet port, a second outlet port, and a second constrained variable volume coupled to the first constrained variable volume. The second constrained variable volume is translatable between a position in fluid communication with the second inlet port and a position in fluid communication with the second outlet port. Furthermore, the second constrained variable volume is configured to expand while in fluid communication with the second inlet port and contract while in fluid communication with the second outlet port. The infusion pump may further include a first fluid reservoir including an interior volume in fluid communication with the first inlet port and a second fluid reservoir including an interior volume in fluid communication with the second inlet port. In some cases, the first constrained variable volume may be configured to expand or contract due to exertion of a translational force through a boundary section of the first constrained variable volume and the second constrained variable volume may also be configured to expand or contract due to exertion of a translational force through a boundary section of the second constrained variable volume.

Some embodiments of an infusion pump include a delivery mechanism having a first delivery section which includes a first inlet port, a first outlet port and a first constrained variable volume that is translatable between a position in fluid communication with the first inlet port and a position in fluid communication with the first outlet port. The delivery mechanism may also include a second delivery section which includes a second inlet port, a second outlet port, and a second constrained variable volume that is translatable between a position in fluid communication with the second inlet port and a position in fluid communication with the second outlet port. The infusion pump may further include a first fluid reservoir including an interior volume in fluid communication with the first inlet port, a second fluid reservoir including an interior volume in fluid communication with the second inlet port and a drive mechanism. The drive mechanism may be operatively coupled to the first constrained variable volume and operatively coupled to the second constrained variable volume. In some such cases, the drive mechanism may be configured to translate the first constrained variable volume between the first inlet port and first outlet port and configured to expand the first constrained variable volume while in fluid communication with the first inlet port and contract the first constrained variable volume while in fluid communication with the first outlet port. The drive mechanism may also be configured to translate the second constrained variable volume between the second inlet port and second outlet port and configured to expand the second constrained variable volume while in fluid communication with the second inlet port and contract the second constrained variable volume while in fluid communication with the second outlet port. In some cases, the drive mechanism may also be configured to expand or contract the first constrained variable volume by exerting translational force through a boundary section of the first constrained variable volume and configured to expand or contract the second constrained variable volume by exerting translational force through a boundary section of the second constrained variable volume.

Some embodiments of an infusion pump for delivering fluids to a patient include a disposable component that includes a delivery mechanism having a first delivery section and a second delivery section. The first delivery section may have a first inlet port, a first outlet port, and a first constrained variable volume that is translatable between a position in fluid communication with the first inlet port and a position in fluid communication with the first outlet port. The second delivery section may have a second inlet port, a second outlet port, and a second constrained variable volume that is translatable between a position in fluid communication with the second inlet port and a position in fluid communication with the second outlet port. The disposable component may further include a first fluid reservoir including an interior volume in fluid communication with the first inlet port and a second fluid reservoir including an interior volume in fluid communication with the second inlet port. The infusion pump may further include a reusable component that includes a drive mechanism which is operatively coupled to the first constrained variable volume and operatively coupled to the second constrained variable volume.

Some embodiments of an infusion pump include a delivery mechanism having a first axial bore section and a first constrained variable volume. The first axial bore section has a first inlet port and a first outlet port in fluid communication with an interior volume of the first axial bore section. The first constrained variable volume includes a maximum axial length less than a distance between the first inlet port and first outlet port. The delivery mechanism also includes a second axial bore section and a second constrained variable volume. The second axial bore section has a second inlet port and second outlet port in fluid communication with an interior volume of the second axial bore section. The second constrained variable volume includes a maximum axial length less than a distance between the second inlet port and second outlet port. The infusion pump may further include a first fluid reservoir including an interior volume in fluid communication with the first inlet port, a second fluid reservoir including an interior volume in fluid communication with the second inlet port and a drive mechanism which is operatively coupled to the first constrained variable volume and which is operatively coupled to the second constrained variable volume.

For some such embodiments, the drive mechanism may be configured to axially translate the first variable volume between the first inlet port and first outlet port and configured to expand the first constrained variable volume while in fluid communication with the first inlet port and to contract the first constrained variable volume while in fluid communication with the first outlet port. The drive mechanism may also be configured to axially translate the second variable volume between the second inlet port and second outlet port and configured to expand the second constrained variable volume while in fluid communication with the second inlet port and contract the second constrained variable volume while in fluid communication with the second outlet port. For these embodiments, the drive mechanism may also be configured to expand or contract the first variable volume by exerting translational axial force through a boundary section of the first variable volume and configured to expand or contract the second variable volume by exerting translational axial force through a boundary section of the second variable volume.

Some embodiments of an infusion pump may include a delivery mechanism having an axial bore. The axial bore may have a first axial bore section, a second axial bore section and a spool disposed in the axial bore. The first axial bore section may include a first inlet port and a first outlet port with said ports being in fluid communication with an interior volume of the first axial bore section. The second axial bore section may include a second inlet port and second outlet port with said ports being in fluid communication with an interior volume of the second axial bore section. The spool may be axially translatable within the axial bore and have a first spool section including a proximal end configured to couple to a drive mechanism and a first seal that forms a fluid tight seal between the first spool section and an interior surface of the axial bore. The first seal is axially fixed relative to the first spool section and slidable relative to the interior surface of the axial bore. The spool may also have a second spool section including a proximal end coupled to a distal end of the first spool section by a limited displacement coupling and a second seal that forms a fluid tight seal between the second spool section and the interior surface of the axial bore. The second seal is axially fixed relative to the second spool section and slidable relative to the interior surface of the axial bore. The spool may also have a third spool section including a proximal end coupled to a distal end of the second spool section by a limited displacement coupling and a third seal that forms a fluid tight seal between the third spool section and the interior surface of the axial bore. The third seal is axially fixed relative to the third spool section and axially slidable relative to the interior surface of the axial bore.

The delivery mechanism may further include a first constrained variable volume and a second constrained variable volume. The first constrained variable volume may be formed between the first spool section, the second spool section, the first seal, the second seal and the interior surface of the axial bore. The second constrained variable volume may be formed between the second spool section, the third spool section, the second seal, the third seal and the interior surface of the axial bore. The infusion pump may further include a first fluid reservoir having an interior volume in fluid communication with the first inlet port, a second fluid reservoir having an interior volume in fluid communication with the second inlet port, and a drive mechanism that is operatively coupled to the proximal end of the spool. In some such embodiments, the drive mechanism may be configured to axially translate the first constrained variable volume between the first inlet port and first outlet port and configured to expand the first constrained variable volume while in fluid communication with the first inlet port and contract the first constrained variable volume while in fluid communication with the first outlet port. The drive mechanism may also be configured to axially translate the second variable volume between the second inlet port and second outlet port and configured to expand the second constrained variable volume while in fluid communication with the second inlet port and contract the second constrained variable volume while in fluid communication with the second outlet port.

Some embodiments of an infusion pump may include a delivery mechanism having an axial bore with a first axial bore section and a second axial bore section. The first axial bore section may include a first inlet port and a first outlet port with said first inlet and first outlet ports being in fluid communication with an interior volume of the first axial bore section. The second axial bore section may be axially spaced from the first axial bore section and may include a second inlet port and second outlet port with said second inlet and outlet ports being in fluid communication with an interior volume of the second axial bore section. The delivery mechanism may also have a spool that includes a proximal end and a distal end. The spool is disposed and axially translatable within the axial bore, and forms a first constrained variable volume between a first seal which is axially fixed relative to the spool, a slidable second seal disposed distally of the first seal, and an interior surface of the axial bore. The spool also forms a second constrained variable volume between a third slidable seal disposed distally of the second slidable seal, a fourth seal disposed distally of the third slidable seal and axially fixed relative to the spool and an interior surface of the axial bore.

The infusion pump may further include a first fluid reservoir having an interior volume in fluid communication with the first inlet port, a second fluid reservoir having an interior volume in fluid communication with the second inlet port and a drive mechanism which is operatively coupled to the proximal end of the spool. In some cases, the drive mechanism may be configured to axially translate the first constrained variable volume between the first inlet port and first outlet port and configured to expand the first constrained variable volume while in fluid communication with the first inlet port and contract the first constrained variable volume while in fluid communication with the first outlet port. The drive mechanism may also be configured to axially translate the second variable volume between the second inlet port and second outlet port and configured to expand the second constrained variable volume while in fluid communication with the second inlet port and contract the second constrained variable volume while in fluid communication with the second outlet port.

Some embodiments of an elastomeric annular seal include an annular seal element which has a substantially uniform cross section along a circumference thereof. The seal element may also include a first annular ring element and a second annular ring element disposed axially adjacent the first ring element with the first and second rings being conjoined or fused by a reduced thickness web there between. The reduced thickness web is configured so as to form an inner annular channel and an outer annular channel between the first ring and second ring. In some cases, an axis of the first ring element and an axis of the second ring element may be separated by a distance equal to about 55 percent to about 70 percent of a transverse dimension of the first and second ring elements.

Certain embodiments are described further in the following description, examples, claims and drawings. These features of embodiments will become more apparent from the following detailed description when taken in conjunction with the accompanying exemplary drawings.

### BRIEF DESCRIPTION OF THE DRAWINGS

FIG. 1 illustrates an embodiment of an interchangeable pump assembly.

FIG. 2 illustrates an embodiment of a full-featured pump device having an infusion cartridge coupled thereto.

FIG. 3 illustrates a block diagram representing an example of a full-featured pump device.

FIG. 4 illustrates an embodiment of an infusion pump system in operative communication with a patient.

FIG. 5 illustrates an enlarged view in partial section of a distal end of an infusion line of the infusion pump system of FIG. 4 disposed subcutaneously in the patient.

FIG. 5A illustrates an enlarged view in partial section of a distal end of an infusion line tubing disposed within and in fluid communication with an interior volume of a vessel of the patient.

FIG. 6 shows a perspective view of an infusion pump embodiment having a graphic user interface with touch screen capability.

FIG. 7 is a rear view of the infusion pump embodiment of FIG. 6 with a disposable infusion cartridge in place.

FIG. 8 illustrates an exploded view of the infusion cartridge and infusion pump embodiment of FIG. 6 with the cartridge removed from the infusion pump.

FIG. 9 is a schematic view partially cut away showing some internal components of the infusion pump embodiment of FIG. 6 and the disposable cartridge.

FIG. 10 is an elevation view in partial section of a delivery mechanism embodiment coupled to a drive mechanism embodiment.

FIG. 11A shows a delivery mechanism embodiment which is configured to deliver fluid from two reservoirs and which is coupled to a drive mechanism embodiment.

FIG. 11AA is an enlarged view in partial section of the delivery mechanism embodiment of FIG. 11A.

FIGS. 11B-11D illustrate a delivery cycle of the delivery mechanism embodiment of FIG. 11A initiating with FIG. 11A.

FIGS. 11E-11H illustrate a delivery cycle of the delivery mechanism embodiment of FIG. 11A.

FIGS. 12A and 12B illustrate a delivery mechanism embodiment that includes a sliding seal configuration.

FIG. 13 illustrates a delivery mechanism embodiment having three outlet ports and three reservoirs in communication with three respective inlet ports.

FIG. 14A illustrates an infusion pump embodiment including a delivery mechanism embodiment having two variable volume elements configured to fill from two separate reservoirs.

FIGS. 14B-14D illustrate a delivery sequence of the variable volume elements of the delivery mechanism embodiment of FIG. 14A, with the delivery sequence initiating at FIG. 14A.

FIGS. 15A-15C illustrate a venting sequence of the delivery mechanism embodiment of FIG. 14A with full variable volume elements.

FIGS. 16A-16B illustrate a delivery sequence of one of the variable volume elements of the delivery mechanism embodiment of FIG. 14A.

FIGS. 16C-16D illustrate a delivery sequence of one of the variable volume elements of the delivery mechanism embodiment of FIG. 14A.

FIGS. 16E-16F illustrate a return sequence embodiment for the delivery mechanism embodiment of FIG. 14A.

FIG. 17A illustrates an infusion pump embodiment having two variable volume elements configured to fill from two separate reservoirs.

FIGS. 17B-17F illustrate a fill and delivery sequence of a variable volume element of the delivery mechanism embodiment of FIG. 17A, with the delivery sequence initiating with FIG. 17A.

FIGS. 18A-18B illustrate a vent sequence for the delivery mechanism embodiment of FIG. 17A.

FIG. 18C illustrates the start of movement from a vent position towards filling a variable volume element of the delivery mechanism.

FIGS. 19A-19F illustrate a fill and delivery sequence of a variable volume element of the delivery mechanism embodiment of FIG. 17A.

FIG. 19G illustrates initiation of a return sequence to a vent position.

FIGS. 20A-20C illustrate a vent sequence of the delivery mechanism embodiment of FIG. 17A.

FIGS. 20D-20E illustrate a return of the variable volume elements to a position where a variable volume element can be filled.

FIG. 21 is a perspective view of an embodiment of an annular seal embodiment.

FIG. 22 is a front view of the annular seal embodiment of FIG. 21.

FIG. 23 is a side view of the annular seal embodiment of FIG. 21.

FIG. 24 is a cross section view of the annular seal of FIG. 23 taken along lines 24-24 of FIG. 23.

FIG. 25 shows a side view of an embodiment of an annular seal embodiment.

FIG. 26 is a front view of the annular seal embodiment of FIG. 25.

FIG. 27 is a cross section view of the annular seal embodiment of FIG. 25 taken along lines 27-27 of FIG. 25.

The drawings illustrate embodiments of the technology and are not limiting. For clarity and ease of illustration, the drawings may not be made to scale and, in some instances, various aspects may be shown exaggerated or enlarged to facilitate an understanding of particular embodiments.

### DETAILED DESCRIPTION

Some infusion device, system, and the method embodiments discussed herein may account for a wide range of variables in determining an amount of medicament, e.g., insulin, to be infused into a patient over a given period of time. Further, some embodiments discussed herein may allow for fine regulation of the amount of medicament delivered as well as the time during which the medicament is delivered. Some embodiments may include advances both in the internal components and the control circuitry as well as improvements in a user interface. The advances may allow for a more accurate regulation of blood glucose levels than is currently attainable by the devices, systems, and methods that are available at this time. Although embodiments described herein may be discussed in the context of the controlled delivery of medicaments such as insulin, other indications and applications are also contemplated. Device and method embodiments discussed herein may be used for pain medication, chemotherapy, iron cleation, immunoglobulin treatment, dextrose or saline IV delivery, or any other suitable indication or application. Non-medical applications are also contemplated.

With regard to one particular indication, maintaining appropriate blood glucose homeostasis is an important factor for promoting the length and quality of life of a diabetic patient. Different types of pumps provide a user with various advantages, some of which can be mutually exclusive. For example, a pump device having a large output display can be easier to read and use compared to a pump device with a smaller output display. But that pump may also have a housing that is generally larger and may require a greater power usage. Large and bulky pump devices can be uncomfortable or unwieldy which can contribute to problems with user compliance. For example, a user may be less likely to wear a larger pump device while sleeping or when involved in certain activities. Smaller and more discreet pump systems known in the art can be more easily worn at night, but do not provide all the features patients have come to rely upon for safety and convenience. And once removed from the skin, known pump devices and their associated insulin cartridges cannot be used again.

In some cases, a single insulin infusion cartridge may be used with a pump device to supply a user with insulin over an extended period of days, such as 3 days. During this time period a user's needs with respect to pump features can change. In some cases full-featured pumps offer certain advantages that a user may not desire at other times such as during sleep or busy weekend activities. Because known insulin cartridges and infusion sets are not interchangeable they cannot be used again once the sterile field is broken and the infusion set and cartridge is used with one pump device. Known infusion sets and insulin cartridges must be thrown out once they are disconnected from a patient.

Some embodiments discussed herein may include an interchangeable pump assembly that provides a user with the flexibility and convenience to alternate between pump devices having various features and advantages at any given moment during a single treatment protocol. In some cases a single insulin cartridge may be switched between pump devices such as a smaller more discreet pumping device having fewer features and a larger full-featured pumping device during a single treatment without compromising the sterility and thus wasting the cartridge.

As discussed above, there are many applications in academic, industrial, and medical fields, as well as others, that may benefit from devices and methods that are capable of accurately and controllably delivering fluids, including liquids and gases that have a beneficial effect when administered in known and controlled quantities. It may be particularly desirable to safely, reliably and comfortably administer required doses of more than one medication or other fluid to a patient from a single device. This may arise in cases where a treatment regimen involving multiple therapeutic substances is coordinated by a single processor or controller which may also be included in a single device, regardless of whether multiple conduits to the patient's body are required.

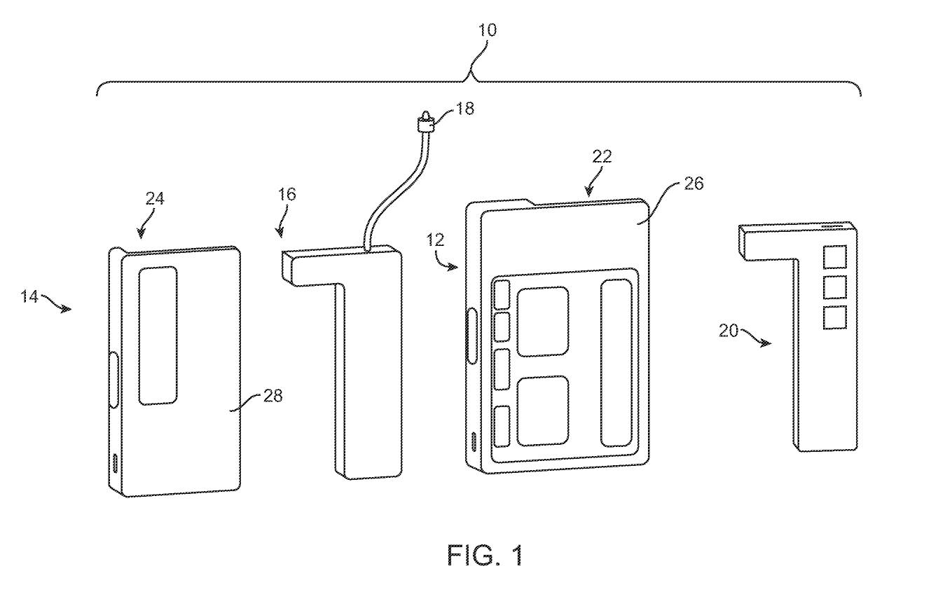

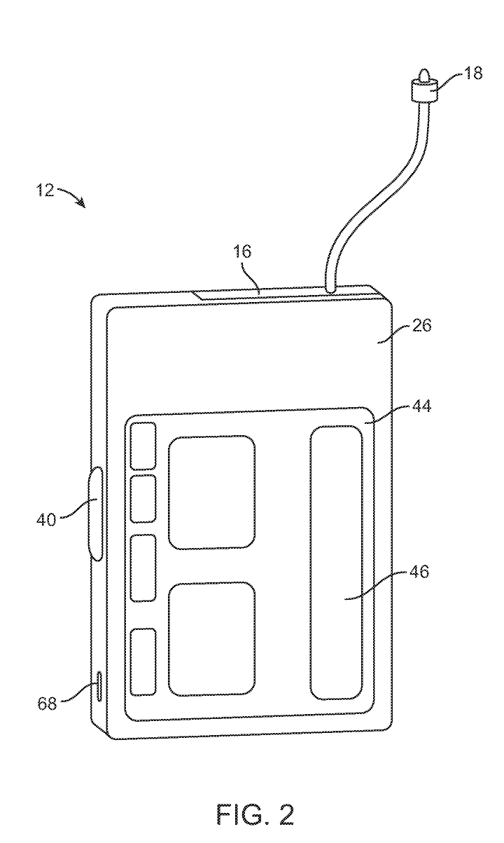

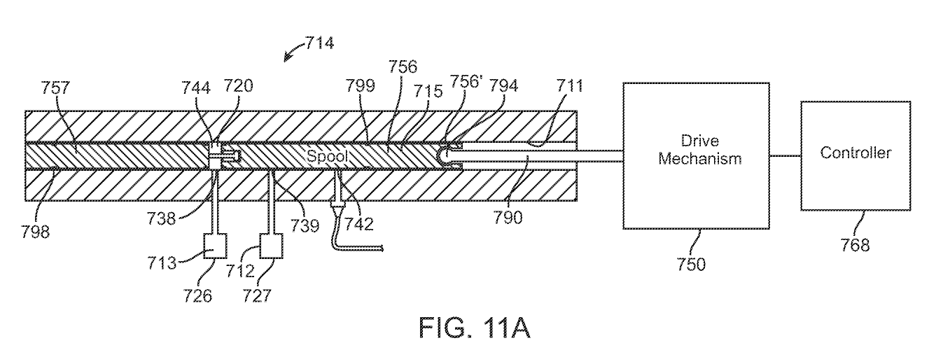

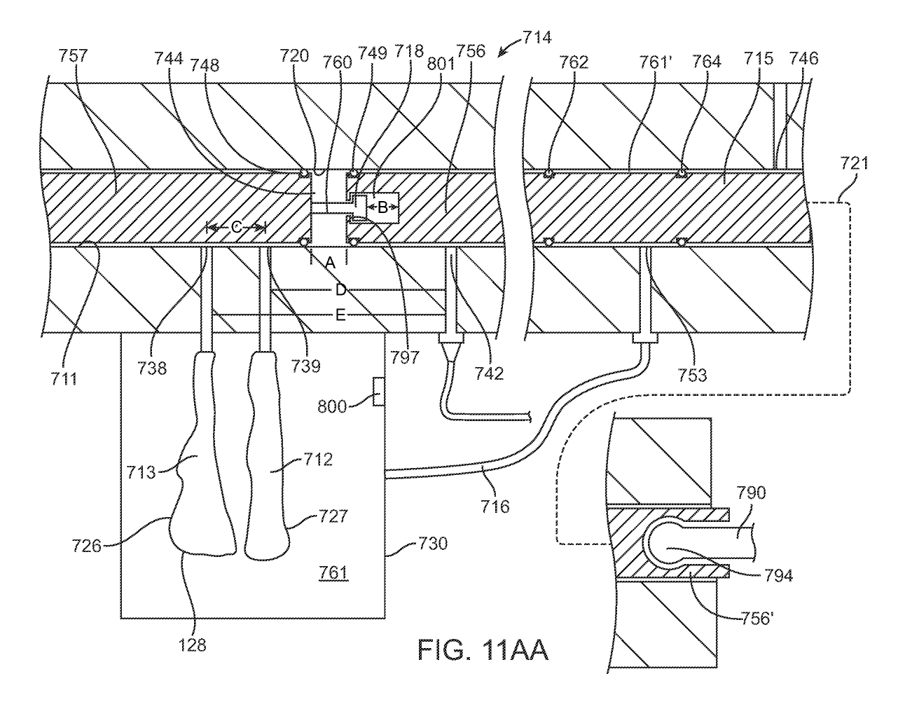

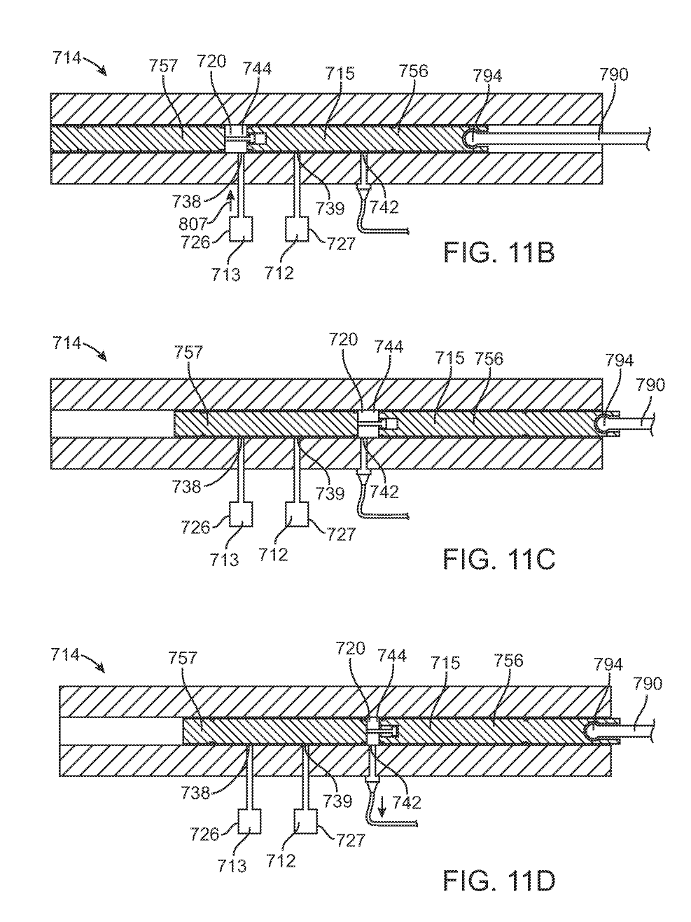

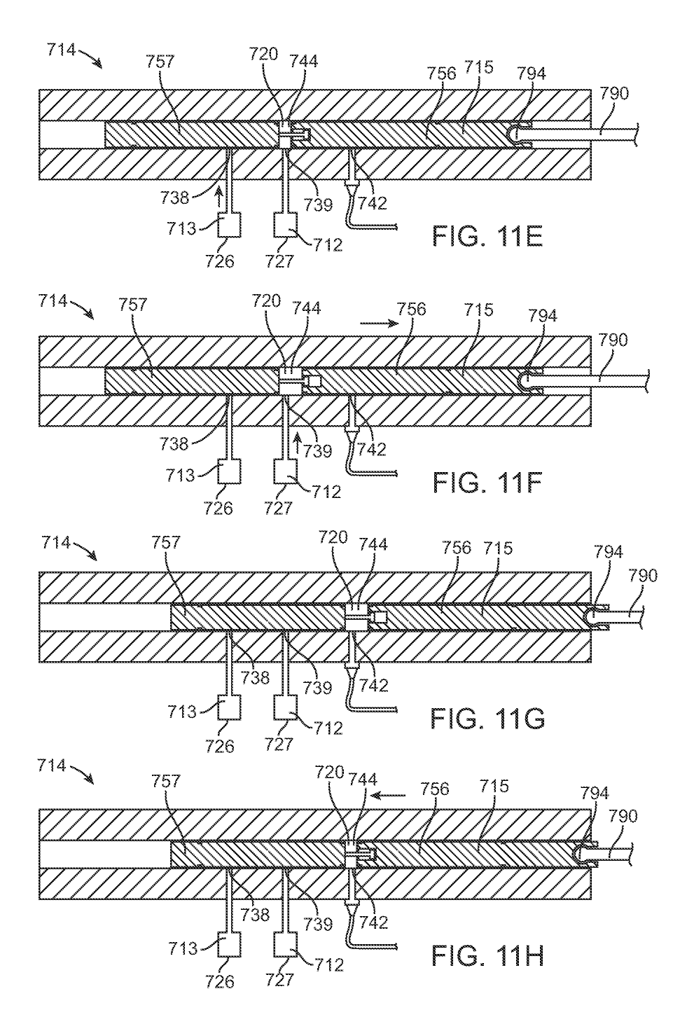

FIGS. 1-3 show an embodiment of interchangeable infusion pump assembly 10 that may be useful for pump embodiments configured to deliver a fluid or fluids from one, two, three or more reservoirs which are in fluid communication with a delivery mechanism thereof. The assembly 10 may include a first pump device 12, a second pump device 14, an infusion cartridge 16 having an infusion set connector 18, and optionally a glucose meter 20. The infusion cartridge may include multiple fluid reservoirs in fluid communication with respective inlet ports as shown in the embodiments of FIG. 11A, 14A or 17A discussed below as well as others. Either the infusion cartridge 16 or the glucose meter 20 may be functionally and interchangeably inserted in a receiving slot 22 located in the first pump 12 and a receiving slot 24 located in the second pump 14. The first pump 12 may have a housing 26 that is generally larger than the housing 28 of the second pump 14 as shown in FIG. 2. Similarly, the first pump 12 generally includes more operating features than the second pump 14. Some or all of the suitable features, dimensions, materials and methods of use of the infusion assembly 10 shown in FIGS. 1-3 may be used or incorporated into any other infusion system, or components thereof, discussed herein. It should also be noted that the interchangeability of infusion cartridge embodiments is discussed herein generally in the context of transferring an infusion cartridge from a first pump to a second pump having features different from those of the first pump. However, all of the interchangeability features and methods associated with this type of transfer may also be applied to the transfer of an infusion cartridge from a first pump to a second pump having the same features as the first pump.

The housing 26 of the first pump device 12 (see FIG. 2) can be of any suitable shape and size. For instance, the housing 26 may be extended and tubular, or in the shape of a square, rectangle, circle, cylinder or the like. The housing 26 may be dimensioned so as to be comfortably associated with a user and/or hidden from view, for instance, within the clothes of a user. In some embodiments, the housing 26 of the full-featured pump device 12 may have a width of about 2 inches to about 5 inches, a height of about 1 inch to about 3 inches and a thickness of about 0.25 inch to about 0.75 inch, more specifically, the housing 26 may have a width of about 2.5 inches to about 3.5 inches, a height of about 1.5 inches to about 2.5 inches and a thickness of about 0.4 inches to about 0.8 inches. The materials of the housing 26 may vary as well. In some embodiments, housing of the full-featured pump device 12 may be a water-tight, metal housing that may be taken apart for repairs.

Still with respect to FIG. 2, the pump device 12 may include an output/display 44. The type of output/display 44 may vary as may be useful for particular application. The type of visual output/display may include LCD displays, LED displays, plasma displays, OLED displays and the like. The output/display 44 may also be an interactive or touch sensitive screen having an input device such as a touch screen, a capacitance screen, a resistive screen or the like. In some embodiments, the output/display 44 of the full-featured pump device 12 may be an OLED screen and the input 40 may be a capacitance touch screen. The pump device 12 may additionally include a keyboard or other input device known in the art for data entry, which may be separate from the display. The output/display 44 of the pump device 12 may also include a capability to operatively couple to a secondary display device such as a laptop computer, mobile communication device such as a smartphone or personal digital assistant (PDA) or the like.

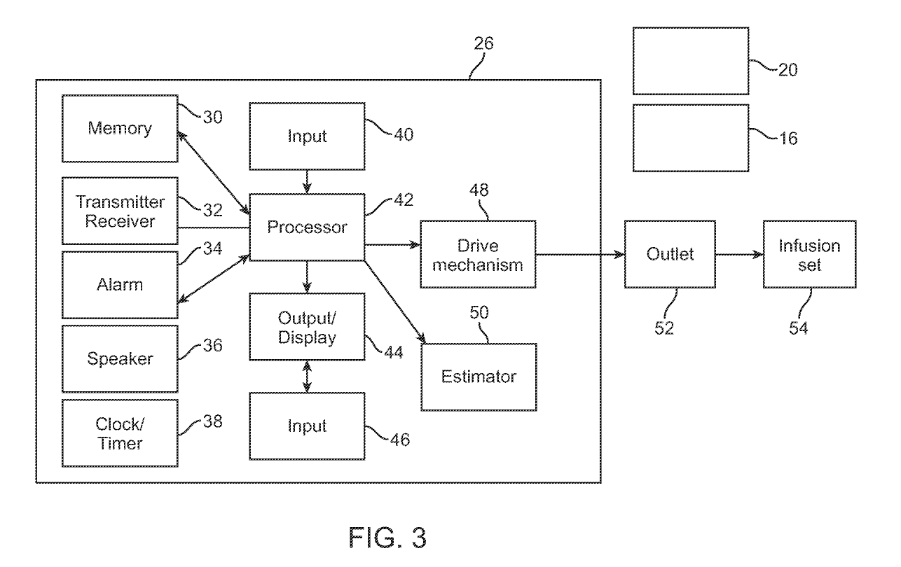

The pump device 12 may have wired or wireless communication capability such as for the sending and receiving of data as is known in the art. The wireless capability may be used for a variety purposes, including updating of any software or firmware for a the processor 42 of the device. The wireless communication capability may vary including, e.g., a transmitter and/or receiver, radiofrequency (RF) transceiver, WIFI connection, infrared or Bluetooth® communication device. The wired communication capability may also vary including, e.g., USB or SD port, flash drive port, or the like. In some embodiments, the first pump device 12 has a transmitter/receiver 32, such as a radiofrequency (RF) transceiver as shown in FIG. 3, that allows the first pump device 12 to communicate with one another and be used interchangeably without loss of data or information during an infusion protocol with a single infusion cartridge 16. The first pump device 12 may also act as a PDA or controller to wirelessly control a second pump device. For such an embodiment, data may be transferred between the controller of the first pump device and second pump device by radio signal, optical transmission or any other suitable means. The first and second pump device 12 may be used as stand-alone devices as well.

One or more of the pump devices 12 may also include GPS functionality, phone functionality, warning and/or alarm programming; music storage and replay functionality, e.g., an MP3 player; a camera or video mechanism; auto scaling capabilities, and/or one or more video type games or other applications developed by third parties for use thereon. One or more of the pump devices 12 may also include an accelerometer, for instance, which may be used for changing presented estimates, wherein instead of scrolling through a menu of options or using a numerical keypad, values can be input or changed via the accelerometer, such as by gesturing with or otherwise shaking the device.

The first pump device 12 may each include a memory device 30. The memory device 30 may be any type of memory capable of storing data and communicating that data to one or more other components of the device, such as the processor. The memory may be one or more of a Flash memory, SRAM, ROM, DRAM, RAM, EPROM, dynamic storage, and the like. For instance, the memory may be coupled to the processor and configured to receive and store input data and/or store one or more template or generated delivery patterns. For example, the memory can be configured to store one or more personalized (e.g., user defined) delivery profiles, such as a profile based on a user's selection and/or grouping of various input factors (as described below); past generated delivery profiles; recommended delivery profiles; one or more traditional delivery profiles, e.g., square wave, dual square wave, basal and bolus rate profiles; and/or the like. The memory can also store user information, history of use, glucose measurements, compliance, an accessible calendar of events, and the like. In some embodiments, the memory 30 of the first pump device 12 may be up to about 10 GB, more specifically, up to about 3 GB, even more specifically, about 1 MB to about 200 MB. In some embodiments, the memory 30 of the first pump 12 may be up to about 3 GB, more specifically, up to about 500 MB, and even more specifically, about 200 kB to about 200 MB.

The first pump device 12 may include a power charging mechanism in some cases, such as a USB port, induction charger, or the like. The power charging system may be used to charge a power storage cell such as a rechargable battery of the pump device. Some embodiments may use a rechargable battery such as a NiCad battery, LiPo battery, NiMH battery or the like. In some embodiments, the power charging mechanism 68 of the first pump 12 shown in FIG. 2 may be a USB port. As such, all data may be kept in the first pump device 12 for quick and easy downloading of data to a computer, other pump device, network etc. using the USB port. The USB port 68 of the first pump device 12 may also provide the first pump device 12 with power charging. In some instances, the power charging mechanism of the second pump device 12 may be an induction charging device. In some cases, an advantage of having interchangeable pumping device 12 may be that while one pump device is being used for infusion, the other pump device can be charging. Further, the use of dual pump device may provide a user of the pump with a back-up in case of failure of one pump device.

The first pump 12 may also include programming to allow processor 42 to make a recommendation regarding a variety of treatment parameters. For instance, the processor 42 may include one or more estimator functionalities 50, which may allow the processor 42 to receive data from various sources, parse the data, collate the same, and generate an estimate based on the same. For instance, the processor 42 may receive user input data and/or data from one or more sensors or other external sources, which the processor 42 can process and thereby use to generate an estimate, such as an estimate of an amount of fluid to deliver to a body, a rate of fluid delivery, and/or a specific fluid delivery profile. For example, the processor 42 may be configured to process data pertinent to a current or predicted condition and to generate an estimate, represented as an amount, rate, profile, etc. of fluid to be delivered based on that data, which estimate may then be displayed to a user, thereby allowing the user to interact with the estimate to accept, decline, and/or otherwise modify the estimate.

As shown in FIG. 3 the first pump device 12 has a processor 42 that functions to control the overall functions of the device. The processor 42 may include programming that functions to control the respective device and its components. The processor 42 may communicate with and/or otherwise control a drive mechanism 48, output/display 44, memory 30, transmitter/receiver 32 and the like. The processor of one of the pump devices may communicate with the processor of the other pump device, for example, through the transmitter/receiver. The processors may include programming that can be run to control the infusion of insulin or other medicament from the cartridge, the data to be displayed by the display, the data to be transmitted via the transmitter, etc. The processors may also include programming that allows the processors to receive signals and/or other data from an input device, such as a sensor that senses pressure, temperature, and the like, that may be included as a part of the device or used in conjunction therewith. The processor 42 may receive signals, for instance, from a transmitter/receiver on the blood glucose monitor and store the signals in the memory.

FIG. 3 illustrates a block diagram of some of the features that may be incorporated within the housing 26 of the first pump 12. The first pump 12 can include a memory device 30, a transmitter/receiver 32, an alarm 34, a speaker 36, a clock/timer 38, an input device 40, a processor 42, an output/display 44 such as a graphic user interface or GUI having an input 46, a drive mechanism 48, and an estimator device 50. As mentioned, the housing 26 of the first pump 12 may be functionally associated with an interchangeable and removable glucose meter 20 or infusion cartridge 16. The infusion cartridge 16 may have an outlet port 52 that may be connected to an infusion set connector 18 and an infusion set 54.

The processor 42 may also include additional programming to allow the processor to learn user preferences and/or user characteristics and/or user history data, for instance, to implement changes in use suggestions based on detected trends, such as weight gain or loss; and may include programming that allows the device to generate reports, such as reports based upon user history, compliance, trending, and/or other such data. Additionally, pump device embodiments of the disclosure may include a “power off” or “suspend” function for suspending one or more functions of the device, such as suspending a delivery protocol, and/or for powering off the device or the delivery mechanism thereof. For some embodiments, two or more processors may be used for controller function of the pumps, including a high power controller and a low power controller used to maintain programming and pump functions in low power mode in order to save battery life.

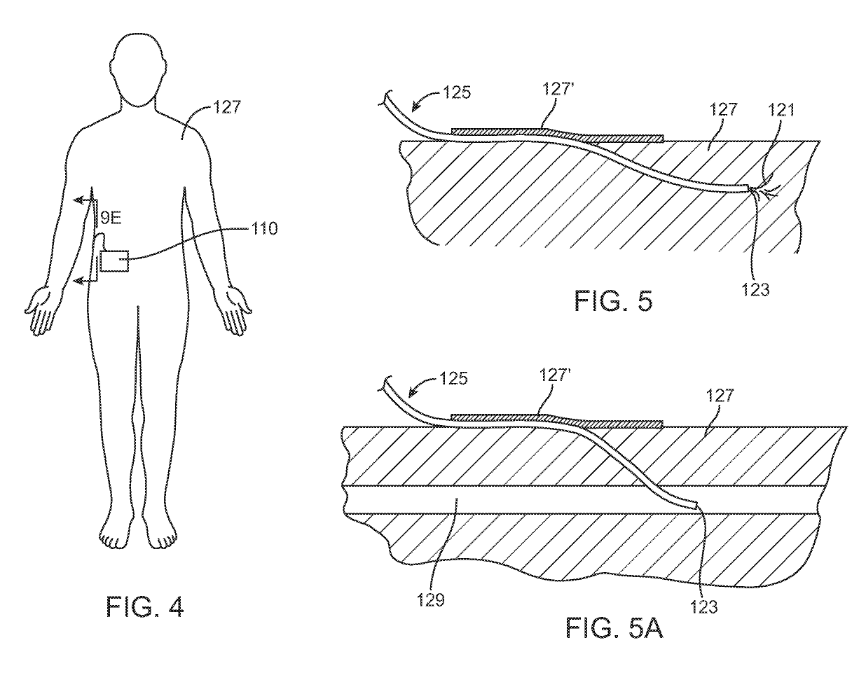

FIG. 4 shows an infusion pump system embodiment 110 operatively coupled to a patient 127. FIG. 5 shows a distal end of an outlet tube 123 of an infusion set 125 disposed beneath the skin of a patient 127. The infusion set 125 is in fluid communication with a dispense part at the pump system 110 and a fluid 121, such as insulin or other suitable medicament, is shown being disposed from the outlet 123 into the body of the patient 127. The distal section of the outlet tube is held in place with a piece of an adhesive pad 127′ secured to the patient's skin 127. The distal end 123 of the infusion set 125 or outlet tube may also be disposed within a patient's fluid vessel 129, such as is shown in FIG. 5A. For such a fluid vessel 129, such as a vein, artery, duct or other, a fluid or multiple fluids 121 may be dispensed from a distal port at a distal end of the outlet tube 123 into interior volume of the patient's vessel 129. The inner lumen of the outlet tube 123 may be in such fluid communication as is shown in FIG. 5 or 5A may also be used in some cases to withdraw fluid from the patient's body 127 back towards the infusion pump 110 for analysis or the like. The proximal end of the infusion set may be in fluid communication with a dispense part such as an outlet port of a delivery mechanism of any of the pump system embodiments discussed herein. Fluids 121, such as insulin or any other suitable medicament, is shown being dispensed from the distal port 123 into the body of the patient 127. As such, any of the infusion pump embodiments discussed herein may be coupled to a patient's body 127 as shown in FIGS. 4-5A.

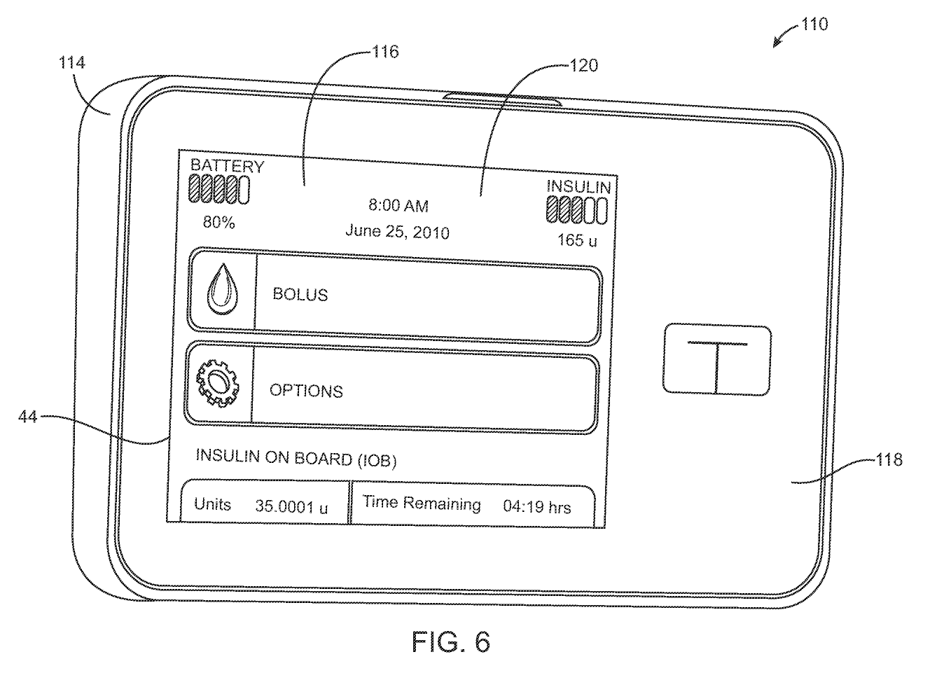

FIGS. 6-10 show an embodiment of an infusion pump system 110, including an infusion cartridge and pump device, which is configured to deliver a fluid from a single fluid reservoir. However, some or all of the features, dimensions or materials of the system 110 shown in FIGS. 6-10 may be incorporated into any other suitable infusion pump system embodiment discussed herein. In addition, where appropriate, any other delivery mechanism embodiment or drive mechanism embodiment discussed herein may be incorporated into the infusion pump system embodiment 110 of FIGS. 6-10. As with previously described embodiments, the infusion cartridge 112 may be a reversibly removable and interchangeable element that may be inserted into different pump devices. The pump device embodiment 114 may have some or all of the same or similar features, dimensions or materials as those of the pump devices illustrated in FIGS. 1-3.

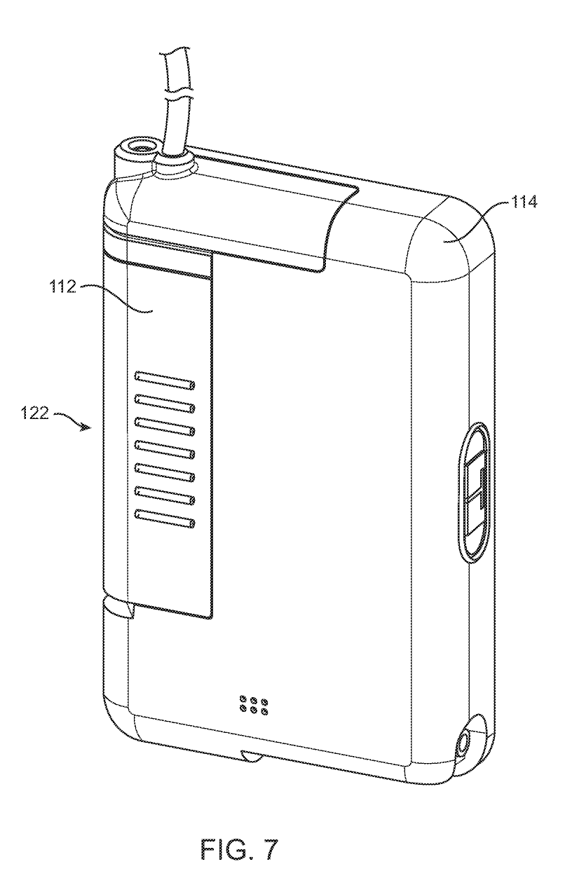

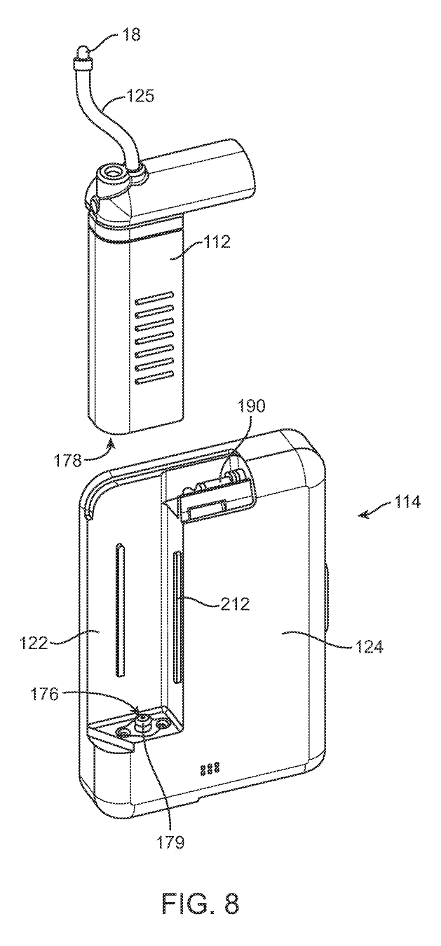

Referring to FIG. 6, a front view of the pump device 114 is shown and includes a user friendly user interface 116 on a front surface 118 of the pump device 114. The user interface 116 includes a touch sensitive screen 120 that may be configured to display a variety of screens used for displaying data, facilitating data entry by a patient, providing visual tutorials, as well as other interface features that may be useful to a patient operating the pump device 114. FIG. 7 shows a rear view of the infusion pump system 110 of FIG. 6 with an infusion cartridge 112 engaged with the pump device 114. FIG. 8 shows the infusion pump system of FIG. 7 with the infusion cartridge 112 detached from the pump device 114 of the system 110.

As shown in FIG. 8, the pump device 114 may include an attachment mechanism 176 positioned within a slot 122 near its terminus that corresponds to a receiving mechanism 178 at an end of the infusion cartridge 112. The attachment and receiving mechanisms may be configured to removably couple an interior volume of the cartridge with a volume of the pump that is sealed from the surrounding environment with the coupling able to retain a fluid within the volumes even under significant pressure. The o-ring based tap attachment embodiment discussed below may be so configured and suitable for producing a leak free detachable coupling that can withstand significant pressure. The receiving mechanism 178 may be configured to detachably couple with the attachment mechanism 176 such that the infusion cartridge 112. The infusion cartridge may be reversibly attached to a housing 124 of the pump device 114 for fluid delivery. In these embodiments, the attachment mechanism 176 may include a pneumatic tap 179 having an O-ring or other sealing device. The corresponding receiving mechanism 178 positioned on an end of the infusion cartridge 112 may include a port through which the pneumatic tap 179 may be inserted.

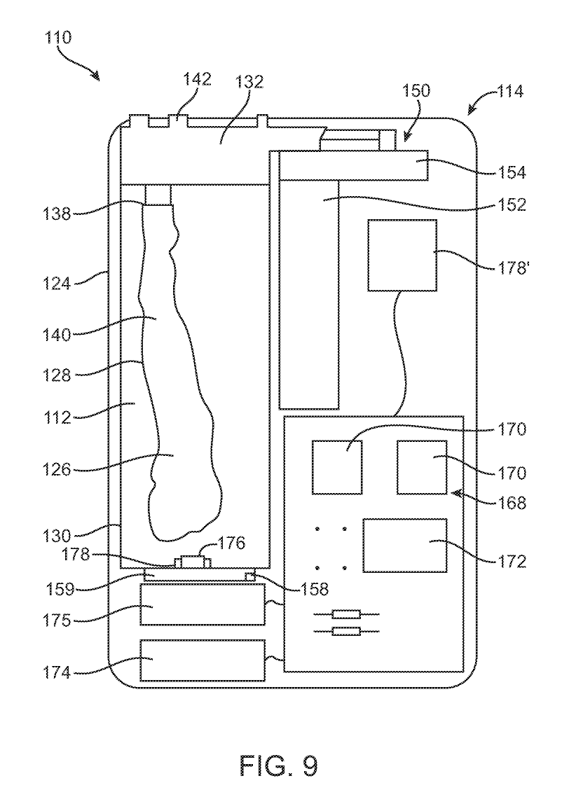

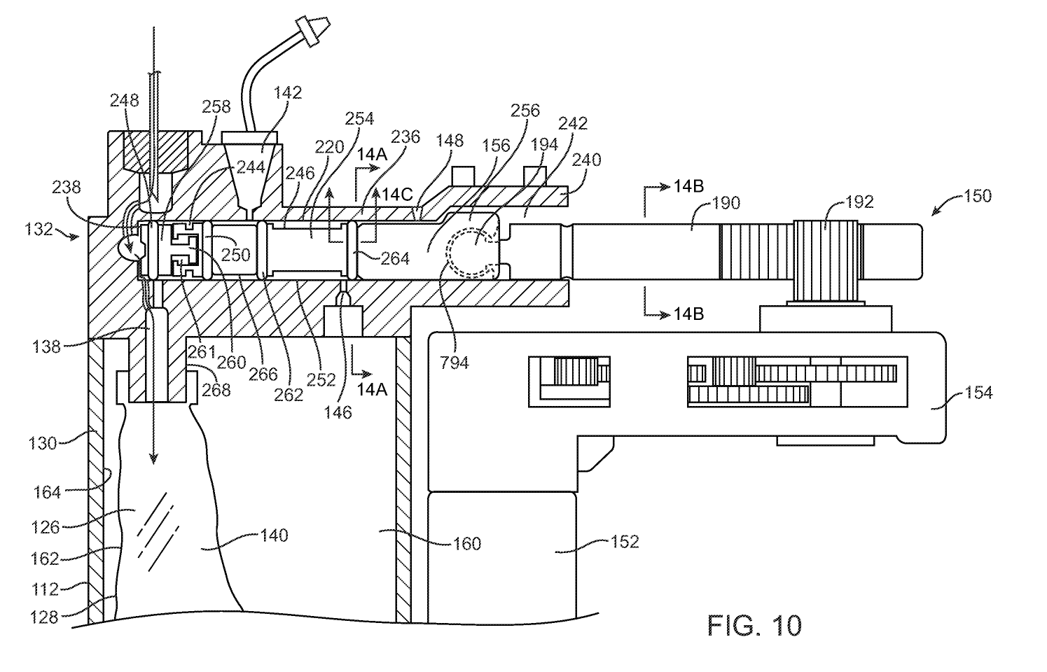

Referring to FIGS. 9 and 10, a collapsible fluid reservoir 126 of the infusion cartridge may be bounded by or disposed within a flexible membrane or layer 128. The fluid reservoir 126 may include an interior volume 140 in fluid communication with a reservoir inlet port 138 of a bore 220 of a delivery mechanism 132. A top portion of the flexible membrane or layer 128 may be clamped or otherwise sealed to an extension or boss 268 of the reservoir inlet port 138 that extends into the cartridge 112. In this configuration, the interior volume 140 of the collapsible fluid reservoir 126 may be isolated or sealed from the surrounding environment except for the reservoir inlet port 138 which is in fluid communication with the bore 220 of the delivery mechanism 132. A substantially rigid shell 130 may be disposed about the collapsible fluid reservoir 126 with an interior volume that contains the collapsible fluid reservoir. The vented volume 160 of the cartridge 112 is disposed between an outer surface 162 of the flexible membrane 128 and an interior surface 164 of the rigid shell 130. The vent inlet port 146 is in fluid communication with the vented volume 160 and the bore 220 of the delivery mechanism 132 as shown in FIG. 10. The vent inlet port 146 is disposed proximally of the reservoir inlet port 138 for the embodiment of the delivery mechanism 132 shown.

As shown in FIG. 9, a graphic user interface (GUI) 116 may be operatively coupled to a controller 168, which may include at least one processor 170, a memory device 172 and connective circuitry or other data conduits that couple the data generating or data managing components of the device. A power storage cell in the form of a battery 174 that may be rechargeable may also be disposed within the housing 124. Data generating or managing components of the device may include the processor(s) 170, the memory device 172, sensors 158, including any pressure or temperature sensors, the GUI 116 and the like. Other components such as a vibratory motor 175, speaker 178′, battery 174 and motor 152 of the drive mechanism 150 may also be operatively coupled to the controller 168. Connective circuitry may include conductive wiring such as copper wiring, fiber optic conduits, RF conduits and the like. For some embodiments, the fluid reservoir cartridge 112, and any of the fluid reservoir cartridges discussed herein, may include an encoder or bar code type strip (not shown). The encoder strip or device may be configured to be scanned and read by a reader device of the pump 114 with the reader device in operative communication with the controller 168 or processor 170 thereof. The encoder device may alternatively be an RFID chip or the like that transmits data to a reader such as a data receiving processor or the like. Such encoder device embodiments may include the ability to securely transmit and store data, such as, via, encryption, to prevent unauthorized access or tampering with such data. The identification of the fluid reservoir cartridge 112 may be used by the controller 168 to set or to adjust certain dispense parameters or any other suitable parameters.

For the embodiment shown, the vent inlet port 146 may be disposed on the delivery mechanism 132 in fluid communication with the vented volume 160 disposed between the outside surface 162 of the flexible material or membrane 128 of the collapsible reservoir 126 and an inside surface 164 of the substantially rigid shell or case 130 of the infusion cartridge. The controller 168 may include at least one processor 170 and a memory device 172, the controller 168 being operatively coupled to the drive mechanism 150, GUI 116, and at least one pressure sensor 158 which may be disposed in communication with a chamber 159 which is in communication with vented volume 160 my means of attachment mechanism 176 and receiving mechanism 178. The controller may be configured to generate a signal to the drive mechanism 150 to displace the spool 156 of the delivery mechanism 132.

FIG. 10 shows a portion of a fluid reservoir cartridge 112 including a delivery mechanism 132 as well as a portion of a drive mechanism of an infusion pump. The delivery mechanism embodiment shown in FIG. 10 is configured to deliver material from a single reservoir 126 through an inlet port 138 to a single outlet port 142 via a collapsible or variable volume element of spool 156. For the embodiments discussed herein, the variable volume elements may include constrained variable volume elements that are mechanically constrained to vary between a minimum volume and a maximum volume. The delivery mechanism 132 includes a delivery mechanism body 236 or housing and a bore 220 disposed in the delivery mechanism body 236. The bore 220, which may have a substantially round transverse cross section, includes a distal end 238, a proximal end 240 disposed towards the drive mechanism 150 of the infusion pump 114, an interior volume 242, a reservoir inlet port 138, a fluid outlet port 142, a vent inlet port 146 and a vent outlet port 148. The spool 156, which may also have a substantially round transverse cross section, is slidingly disposed within the bore 220 and forms a constrained variable first volume 244 and a vent second volume 246 with the bore 220.

The constrained variable first volume 244 of the delivery mechanism 132 may be positionable to overlap the reservoir inlet port 138 independent of an overlap of the fluid dispense port 142. The variable first volume 244 may be formed between a first seal 248 around the spool 156, a second seal 250 around the spool 156, an outer surface 266 of the spool body between the first and second seal 248 and 250 and an interior surface 252 of the bore 220 between the first and second seal 248 and 250. The first and second seals 248 and 250 are axially moveable relative to each other so as to increase a volume of the variable volume 244 when the first and second seals 248 and 250 are moved away from each other and decrease the variable volume 244 when the seals 248 and 250 are moved closer together.

The second seal 250 is disposed on a main or proximal section 254 of the spool 156 and moves in conjunction with movement of the rest of the spool. A proximal end 256 of the spool 156 is coupled to a ball portion 194 of a drive shaft 190 of the drive mechanism 150 of the pump device 114. The drive mechanism 150 includes a rack and pinion mechanism 192 actuated by an electric motor 152 through a gear box 154. As such, the second seal 250 moves or translates axially in step with axial translation of the spool 156 and drive shaft 190. The first seal 248, however, is disposed on a distal section 258 of the spool 156 which is axially displaceable with respect to the main section 254 of the spool 156. The distal section of the spool 156 is coupled to the main section of the spool by an axial extension 260 that is mechanically captured by a cavity 261 in the main section 254 of the spool 156. This configuration allows a predetermined amount of relative free axial movement between the distal section 258 of the spool and the nominal main section 254 of the spool 156.

For some embodiments, a volume of a “bucket” of fluid dispensed by a complete and full dispense cycle of the spool 156 may be approximately equal to the cross section area of the bore 220 multiplied by the length of displacement of the captured axial extension of the spool for the distal section 258. The complete bucket of fluid may also be dispensed in smaller sub-volumes in increments as small as a resolution of the drive mechanism 150 allows. For some embodiments, a dispense volume or bucket defined by the complete variable volume 244 of the delivery mechanism 132 may be divided into about 10 to about 100 sub-volumes to be delivered or dispensed. In some cases, the maximum axial displacement between the distal section and main section of the spool may be about 0.01 inch to about 0.04 inch, more specifically, about 0.018 inch, to about 0.022 inch.

For some embodiments, the bore 220 of the delivery mechanism may have a transverse dimension or diameter of about 0.04 inches to about 0.5 inches, more specifically, about 0.08 inches to about 0.15 inches. For some embodiments, the spool 156 may have a length of about 10 mm to about 40 mm, more specifically, about 15 mm to about 20 mm. The spool 156 and housing of the delivery mechanism 132 may be made from any suitable material or materials including polymers or plastics such as polycarbonate, PEEK, thermoplastics, cyclic olefin copolymer, and the like. In some cases, the seals disposed on the spool may have an outer transverse dimension or diameter that is slightly larger than that of the spool 156. In some instances, the seals on the spool may have an axial thickness of about 0.01 inches to about 0.03 inches and may be made from materials such as butyl, silicone, polyurethanes or the like having a shore hardness of about 65 A to about 75 A, more specifically, about 70 A.

In some instances, a vent second volume 246 of the delivery mechanism 132 may be formed by the spool 156 and bore 220 of the delivery mechanism 132. The vent second volume 246 is also be formed by a third seal 262 disposed around the spool 156 and a fourth seal 264 also disposed around the spool and axially separated from the third seal 262. The axial separation between the third and fourth seals 262 and 264 forming the vent second volume 246 may be greater than the axial separation between the vent inlet port 146 and vent outlet port 148 of the bore 220 in some instances. The vent second volume 246 is also formed by an outside surface 266 of the spool 156 between the third and fourth seal 262 and 264 an inside surface 252 of the bore 220 between the third and fourth seal 262 and 264.

The vent second volume 246 may be axially displaceable with the movement of the spool 156 and may also be positionable by such axial displacement in order to simultaneously overlap the vent second volume 246 with the vent inlet port 146 and vent outlet port 148 of the bore 220. Such an overlap of both the vent inlet port 146 and vent outlet port 148 puts these ports in fluid communication with each other and allows an equilibration of pressure between the vented volume 160 of the reservoir cartridge 112 and the environment surrounding the vent outlet port 148. In most cases, the vent outlet port 148 will be in communication with the atmosphere and air will pass from the environment surrounding the vent outlet port 148, through the vent second volume 246 of the bore 220 and into the vent volume 160 to replace the fluid dispensed subsequent to the last vent cycle. When the vent inlet port 146 and vent outlet port 148 do not share a common volume formed by the spool and bore of the delivery mechanism 132, they are typically isolated and no venting of the vented volume takes place.

Devices and methods for measuring and/or confirming a volume of material dispensed and the like from a delivery mechanism or flow metering device are discussed in co-pending, commonly owned U.S. patent application Ser. No. 12/714,299, filed Feb. 26, 2010, by M. Rosinko et al., titled Methods and Devices for Determination of Flow Reservoir Volume, which is incorporate by reference herein in its entirety. The methods and devices discussed therein include measuring a pressure increase in a vented volume of a fluid reservoir cartridge between the rigid shell and flexible membrane of the fluid reservoir as discussed herein. Such pressure measurements may be used to determine or confirm an amount of fluid dispensed, as well as detect malfunctions in the components of a delivery mechanism or drive mechanism of a pump system.

Other methods and devices used for calculating and measuring flow volumes dispensed are discussed in U.S. Pat. No. 7,008,403, filed on Jul. 19, 2002, by Scott Mallett, titled Infusion Pump and Method for Use, U.S. Pat. No. 7,341,581, filed on Jan. 27, 2006, by Scott Mallet, titled Infusion Pump and Method for Use, U.S. Pat. No. 7,374,556, filed on Jan. 31, 2006, by Scott Mallett, titled Infusion Pump and Method for Use, 2007/0264130, filed on May 4, 2007, by Scott Mallett, titled Infusion Pumps and Method for Use, and 2009/0191067, filed on Jan. 25, 2008, by Paul DiPerna, titled Two Chamber Pumps and Related Methods, which are all incorporated by reference herein in their entirety. Some embodiments discussed in these references include the use of the ideal gas law or Boyle's law, for determination of a volume of material dispensed from a device or reservoir thereof. Such methods and devices may be used in conjunction with or as part of suitable embodiments discussed herein.

In operation, the spool 156 and the particular volumes formed between the spool 156, the bore 220 and the circumferential seals 248, 250, 262 and 264 disposed on the spool of the delivery mechanism 132 are typically translated in a proximal and distal direction in order to move the volumes into and out of communication with the various ports of the bore 220. This axial movement in alternating proximal and distal directions of the spool 156 within the bore 220 may be used to put the various ports in fluid communication with translatable volumes of the delivery mechanism 132 and other ports of the mechanism. For reliable operation, it may be desirable in some circumstances for the spool 156 and the circumferential seals 248, 250, 262, 264 disposed about the spool 156 to move smoothly within the bore 220 of the delivery mechanism 132 while maintaining a seal between an outside surface 266 of the spool 156 and an inside surface 252 of the bore. It may also be desirable for the seals 248, 250, 262, and 264 disposed on the spool 156 to move axially back and forth within the bore 220 while maintaining a seal and with a minimum of friction. Achieving these features of the spool 156 may be facilitated with the use of particular seal configurations or gland configurations used to house the seals of the spool embodiments.

Although the delivery mechanism embodiment shown in FIGS. 6-10 is shown configured to deliver fluid(s) from a single reservoir to a single outlet port, a similar arrangement may be used for delivering fluids from multiple reservoirs to one or more outlet ports. An embodiment of a delivery mechanism configuration suitable for delivering fluids from multiple fluid reservoirs to one or more outlet ports is shown in FIGS. 11AA and 11A-11D. The delivery mechanism 714 of FIG. 11AA may be used to deliver fluids from multiple reservoirs with a single spool 756 driven by a single drive mechanism 750 including a motor which may be controlled by a controller 768. Use of a single spool 756 and drive mechanism 750 may be useful with regard to cost of the device, an efficient use of stored energy or battery life and overall size and weight of the device. The drive mechanism 750 used in some cases may be the same as or similar to the drive mechanism 150 of the infusion pump system 110 shown in FIGS. 6-10 and discussed above. As such, the proximal end 757 of the spool 756 of the delivery mechanism 714 in FIG. 11A may include a coupling, such as a ball coupling 794, that is suitable for releasable connection to a distal end of a drive shaft 790 such as drive shaft 190 shown in the embodiment of FIGS. 6-10. The delivery mechanism 714 of FIG. 11A may also have any other features, dimensions, modes of operation or materials which are the same as or similar to those of the delivery mechanism 150 embodiment shown in FIGS. 6-10. In some cases, the spool 756 may be actuated by a drive mechanism 750 including a motor powered by a power storage cell and controlled by the controller 768.

Referring again to FIG. 11AA, in some cases an infusion pump system, similar to 110 shown in FIGS. 6-10, may include the drive mechanism 750 discussed above including a motor, reduction gears and drive shaft 790 coupled to the spool 756 of the delivery mechanism 714 shown in FIG. 11AA. The delivery mechanism 714 may be disposed within a housing of the infusion pump and include an axial bore 720 with a longitudinal axis 721 and an interior volume. A first inlet port 738 is disposed in fluid communication with the interior volume of the axial bore 720. A second inlet port 739 which is axially spaced from the first inlet port 738 is also in fluid communication with the interior volume of the axial bore 720. At least one outlet port 742, which is axially spaced from the first inlet port 738 and second inlet port 739, may also be disposed in fluid communication with the interior volume of the axial bore 720. The spool 756 which is disposed within the axial bore 720 may be axially translatable within the axial bore 720 and forms a constrained variable volume 744 in conjunction with an interior surface 711 of the axial bore 720. The pump system may also include a first fluid reservoir 726 which has an interior volume 713 in fluid communication with the first inlet port 738 and a second fluid reservoir 727 which also has an interior volume 712 in fluid communication with the second inlet port 739. In some cases, both the first and second fluid reservoirs 726 and 727 may be configured as collapsible reservoirs bounded by a thin flexible fluid tight material 128 as shown in the embodiment illustrated in FIGS. 9 and 10. For the embodiment shown, a substantially rigid fluid tight shell 730 may be disposed about the first and second fluid reservoirs 726 and 727 with a fluid tight interior volume 761 being formed between an inside surface of the rigid shell 730 and respective outside surfaces of the first and second fluid reservoirs 726 and 727.

The interior volume 761 may be formed between an inside surface of the substantially rigid shell 730 and outer surfaces of the collapsible flexible fluid reservoirs 726 and 727 and in some cases, this interior volume 761 may include a vented volume 761. The vented volume 761 may be controllably vented by the delivery mechanism 714 with a vent volume 761′ formed between two seals 762 and 764 disposed on the first or proximal spool section 715 of the spool 756. The vent volume 761′ on the spool 756 may be translatable to place a vent inlet port 753 of the axial bore 720 in fluid communication with a vent outlet port 746 of the axial bore 720. A conduit 716 having an inner lumen 717 may be used to communicate between the vented volume 761 and the vent inlet port 753.

In general, in order to deliver fluids from the first fluid reservoir 726 and second fluid reservoir 727, the drive mechanism 750 may be configured to axially translate the constrained variable volume 744 from each of the inlet ports 738 and 739 to the outlet port 742 shown. The drive mechanism 750 may also be configured manipulate the spool 756 or components thereof in order to expand or contract the constrained variable volume 744 of the spool 756 by exerting translational axial force through a boundary section of the constrained variable volume 744. Such an operational configuration of the drive mechanism 750 may be controlled by the controller 768 which is operatively coupled to the drive mechanism 750, and in some cases, the motor of the drive mechanism 750.

As shown in FIG. 11AA, the constrained variable volume 744 is formed between a first seal 749 disposed around a first section or proximal section 715 of the spool 756 and a second seal 748 is disposed around a second section or distal section 757 of the spool 756. The first spool section 715 is coupled to the drive shaft 790 of the drive mechanism 750 at a proximal end thereof. The first section 715 of the spool 756 and second section 757 of the spool 756 are coupled together by a limited displacement coupling which is configured to allow axial movement of the first section of the spool 715 relative to the second section 757 of the spool 756 over a limited axial distance. The limited displacement coupling provides an arrangement whereby the first seal 749 and second seal 748 are axially translatable relative to each other but mechanically constrained to a maximum and minimum axial separation. The limited displacement coupling embodiment shown in FIG. 11AA includes an extension member 760 that extends proximally from the second spool section 757 and includes an enlarged portion or “T” shape 718 at a proximal end of the extension member 760. The extension 760 extends into a cavity of the proximal spool section 715 with the enlarged portion 718 of the extension being axially translatable within the cavity by mechanically captured by a shoulder portion 797 at a distal end of the cavity.

With the configuration of the constrained variable volume 744 shown, the constrained variable 744 volume may expand to a maximum volume when the first seal 749 of the proximal spool section 715 and the second seal 748 of the distal spool section 757 are positioned at a maximum axial separation as illustrated in FIG. 11AA. In this position, the first and second seals 748 and 749 which define moveable boundaries of the constrained variable volume 744 are as axially separated as allowed by the limited displacement coupling. As shown, the enlarged portion 718 of the extension 760 of the limited displacement coupling is adjacent the shoulder portion 797 of the cavity and prevents any further axial separation. In addition, the constrained variable volume 744 of the spool 756 is bounded by an inside surface of the bore 720, an outside surface of a proximal end of the distal or second spool section 757, the second seal 748, an outside surface of a distal end of the first or proximal spool section 715 and the first seal 749. The first seal 749 is sealed against the outside surface of a distal end of the first or proximal spool section 715 and is substantially axially fixed relative to the first spool section 715. The first seal 749 also forms a fluid tight seal against an inside surface of the axial bore 720, but is slidable relative to the inside surface of the axial bore 720. As such, the first seal 749 forms a fixed seal relative to the first spool section 715 and a moveable or axially translatable seal relative to the axial bore 720 of the delivery mechanism 714. The second seal 748 is sealed against the outside surface of a proximal end of the second or distal spool section 757 and is substantially axially fixed relative to the second spool section 757. The second seal 748 also forms a fluid tight seal against an inside surface of the axial bore 720, but is slidable relative to the inside surface of the axial bore 720. As such, the second seal 748 forms a fixed seal relative to the second spool section 757 and a moveable or axially translatable seal relative to the axial bore 720 of the delivery mechanism. Each of the first seal and second seal 748 and 749 are axially translatable relative to the inner surface of the axial bore 720, however, the outward pressure of the seals which may be necessary to provide a fluid tight seal, produces some friction which resists the axial displacement of the seals relative to the inner surface of the axial bore 720. Generally, the spool sections 715 and 757 and seals 748 and 749 of the spool 756 remain stationary unless an external axial force is applied, typically by the drive mechanism through the drive shaft 790 and first spool section 715.

For the embodiment of the constrained variable volume 744 shown, the constrained variable volume 744 may be expanded or contracted by exertion of axial force through a boundary section of the constrained variable volume 744. In particular, when the drive mechanism imparts axial force on the proximal section 715 of the spool 756, that force may be exerted against the contents of the constrained variable volume 744 by the distal end of the first spool section 715 and first seal 749. The distal end of the first spool section 715 and first seal 749 on the first spool section 715 form a boundary section of the constrained variable volume 744 and axial force from the drive mechanism 750 may be applied to the contents of the constrained variable volume 744 in this way. Resulting axial force may thus be applied to the second spool section 757 by increased pressure of the contents of the constrained variable volume 744 pushing against the proximal end of the second spool section 757 and second seal 748, which also may be considered to form a boundary section of the constrained variable volume 744. If the constrained variable volume 744 is in fluid communication with an inlet port 738 or 739 or outlet port 742, the increased pressure or decreased pressure resulting from axial force being applied to the first spool section 715 by the drive mechanism 750 may cause the constrained variable volume 744 to expand or contract. Such an action may thus draw fluid into the expanding constrained variable volume 744 or deliver fluid from a contracting constrained variable volume 744.

The maximum delivery pressure that may be generated from the spool 756 embodiment shown in FIG. 11AA may be determined by the maximum force that the drive mechanism may generate as well as the maximum frictional resistance to axial movement produced by the second seal 748 (or any other moveable seals) of the second spool section 757 against the inner surface of the axial bore 720. As such, the maximum pressure that may be generated from the pump action of the delivery mechanism 714 may be about equal to the force of frictional resistance to axial translation of the second spool section 757 divided by the cross sectional area of the axial bore 720. As such, it may be desirable in some cases to select the size and type of seal for the spool 756 in order to achieve a desired level of maximum delivery pressure of the device.

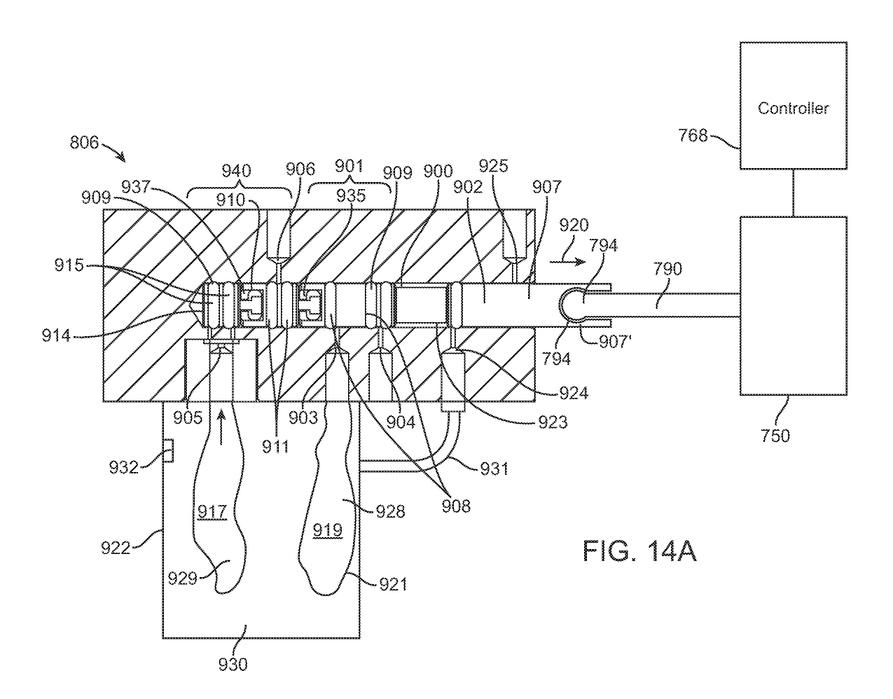

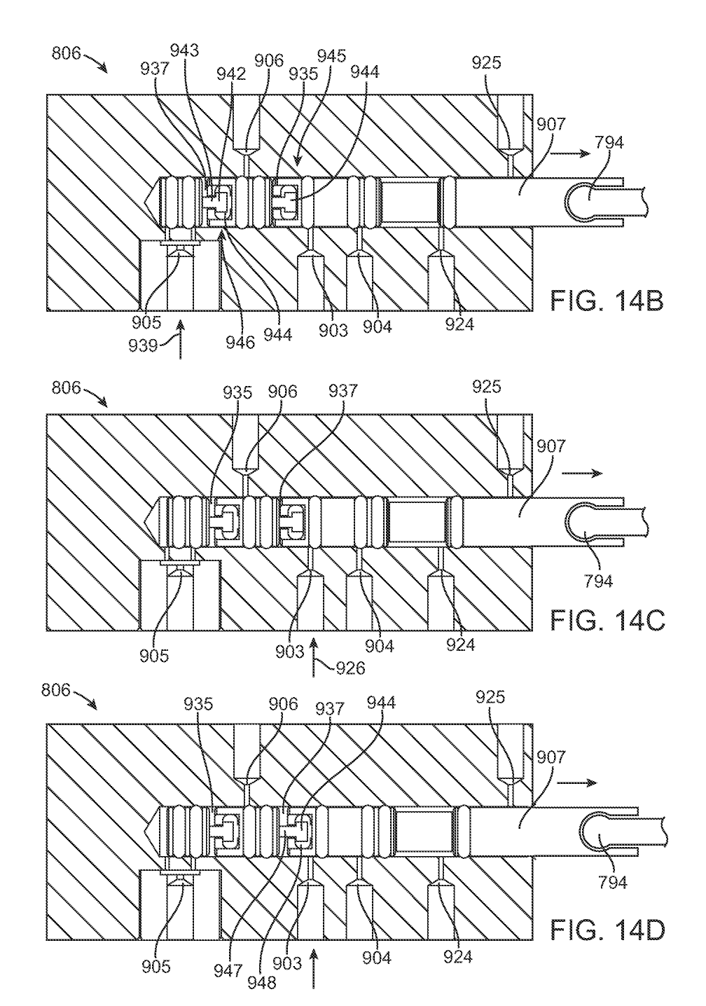

In order to separate the drawing and dispensing or delivering functions of the constrained variable volume 744, it may be necessary to configure the separation and location of the various inlet 738 or 739 and outlet ports 742. For some delivery mechanism embodiments 714, it may be desirable for an axial separation of the first inlet port 738 from the second inlet port 739, indicated as C in FIG. 11AA, to be greater than a maximum axial length of the constrained variable volume 744, which is indicated as A in FIG. 11AA. In addition, it may also be desirable for an axial separation of the proximal most inlet port 739 from the distal most outlet port 742, indicated by D in FIG. 11AA, to be greater than a maximum axial length A of the constrained variable volume 744. As shown in FIG. 14A, the bore may also include a plurality of inlet ports or in fluid communication with a single fluid reservoir. This may be desirable in cases where a seal of the spool must be configured to translate axially to a closed end of the axial bore in order to prevent hydraulic locking of the spool movement. In some such circumstances, the delivery mechanism may be configured such that the constrained variable volume is positionable to overlap all of the plurality of inlet ports of this single fluid reservoir independent of an overlap with an inlet port in fluid communication with another fluid reservoir. It may also be acceptable for some of the multiple inlet ports to be isolated if the constrained variable volume is not in an overlap with the inlet port. Some delivery mechanism embodiments may also include a distal bore seal 798 and a proximal bore seal 799 disposed between an outside surface of a spool section and the inside surface of the axial bore at respective proximal and distal ends of the spool. The bore seals 798 and 799 may be configured to isolate the inlet and outlet ports of the bore from the surrounding environment at all times while still allowing axial movement of the spool or spool sections.

For some embodiments, a volume of a “bucket” of fluid dispensed by a complete and full dispense cycle of the spool 756 may be approximately equal to the cross section area of the bore 720 multiplied by the length of displacement of the captured axial extension of the spool 756 for the distal section 757. The complete bucket of fluid may also be dispensed in smaller sub-volumes in increments as small as a resolution of the drive mechanism allows. For some embodiments, a dispense volume or bucket defined by the complete variable volume of the delivery mechanism 714 may be divided into about 10 to about 100 sub-volumes to be delivered or dispensed. In some cases, the maximum axial displacement between the distal section 757 and proximal section 715 of the spool 756 may be about 0.01 inch to about 0.04 inch, more specifically, about 0.018 inch, to about 0.022 inch.

For some embodiments, the bore 720 of the delivery mechanism 714 may have a transverse dimension or diameter of about 0.04 inches to about 0.5 inches, more specifically, about 0.08 inches to about 0.15 inches. For some embodiments, the spool 756 may have a length of about 10 mm to about 40 mm, more specifically, about 15 mm to about 20 mm. The spool 756 and housing of the delivery mechanism 714 may be made from any suitable material or materials including polymers or plastics such as polycarbonate, PEEK, thermoplastics, cyclic olefin copolymer, and the like. In some cases, the seals disposed on the spool 756 may have an outer transverse dimension or diameter that is slightly larger than that of the spool 756. In some instances, seals 748, 749, 762, and 764 on the spool 756 may have an axial thickness of about 0.01 inches to about 0.03 inches and may be made from materials such as butyl, silicone, polyurethanes or the like having a shore hardness of about 65 A to about 75 A, more specifically, about 70 A.

As discussed above, the fluid reservoirs 726 and 727 may be disposed in a substantially rigid shell 730 having an interior volume 761 that may be a vented volume. When venting of the vented volume is desirable, the controller 768 may be configured to deliver a drive signal to the drive mechanism 750 so as to axially translate the spool 756 to a position whereby the vent volume 761′ of the spool 756 is in fluid communication with the vent inlet port 753 and vent outlet port 746. In this position, the vented volume 761 is put into fluid communication with the ambient atmosphere or any other desirable or predetermined environment. In addition, a pressure sensor 800 may be disposed within the vented volume 761 or in fluid communication with the vented volume 761 to determine the pressure within the vented volume 761. Such pressure measurements may be useful for determining the amount of fluid dispensed from the fluid reservoirs 726 or 727, determining when venting of the vented volume 761 is necessary, detecting leaks or clogs in the infusion pump system or the like.

In use, the infusion pump system 710 represented by the embodiment shown in FIG. 11AA may be used to deliver a single type of fluid or two different types of fluid at a controllable and predetermined rate to a patient or a desired receptacle other than a patient. Referring to the delivery sequence illustrated in FIGS. 11A-11D, the delivery process or dispense cycle for delivering fluids from an interior volume 712 and 713 of the first and second fluid reservoirs 726 and 727 may be initiated by translating the constrained variable volume 744 into a position in fluid communication with the second or distal inlet port 738 as shown in FIG. 11A. This position may be achieved by applying a distal axial force from the drive shaft 790 to the first spool section 715. The distally oriented axial force exerted by the drive mechanism 750 is then applied to the second spool section 757 through the limited displacement coupling which will already be axially contracted if a dispense cycle preceded the dispense cycle. In this position with the constrained variable volume 744 in a contracted state and in fluid communication with the second inlet port 739, translational axial force in a proximal direction may be exerted through a boundary section on the proximal spool section 715 of the constrained variable volume 744 to expand the constrained variable volume 744 and draw fluid into the constrained variable volume 744 through the second inlet port 739 from the second reservoir 727 as shown in FIG. 11B. More specifically, the drive shaft 790 of the drive mechanism 750 moves in a proximal direction which, in turn, moves the first or proximal spool section 715 in a proximal direction. The drive mechanism 750 moves the proximal spool section 715 in a proximal direction by overcoming the frictional force between the seal or seals of the proximal spool section 715 against the inner surface 711 of the axial bore 720. As the proximal spool section 715 begins to move proximally away from the distal spool section 757, the constrained variable volume 744 begins to expand and draw fluid into the volume from the second fluid reservoir 727 as shown by the arrow 807 in FIG. 11B.

This process of drawing fluid into the constrained variable volume 744 will occur so long as the force or pressure differential required to draw fluid from the first fluid reservoir 726 is less than the frictional force generated by the seal or seals of the second spool section 757 against the inner surface of the axial bore 720. If the force required to draw the fluid from the first fluid reservoir 726 is too high (such as might be caused by a clog in the inlet port), the second spool section 757 may then be pushed proximally by atmospheric pressure against a distal end thereof and follow the proximal spool section 715 in a proximal direction before the constrained variable volume 744 is full. Otherwise, the constrained variable volume 744 will continue to expand and fill from the first fluid reservoir 726 through the first inlet port 738 until the expanded portion of the extension 760 of the limited displacement coupling is disposed in contact with the shoulder portion 797 of the cavity 801 as shown in FIG. 11B. Once the constrained variable volume 744 is full, the constrained variable volume 744 may then be translated into a position in fluid communication with the outlet port 742 as shown in FIG. 11C. Once so positioned, translational axial force may be applied through the boundary section of the constrained variable volume 744 so as to at least partially contract the constrained variable volume 744 and dispense fluid from the constrained variable volume 744 through the outlet port 742 and to a patient. More specifically, the drive shaft 790 of the drive mechanism 750 moves in a distal direction which, in turn, moves the first or proximal spool section 715 in a distal direction. The drive mechanism 750 moves the proximal spool section 715 in a distal direction by overcoming the frictional force between the seal or seals of the proximal spool section 715 against the inner surface of the axial bore 720. As the proximal spool section 715 begins to move distally towards the distal spool section 757, the constrained variable volume 744 begins to contract and deliver fluid into the outlet port 742. As discussed above, the maximum dispense pressure may be determined at this step by the maximum distal axial force that may be generated by the drive mechanism 750 as well as the frictional force between the seal or seals of the distal spool section 757 and the inner surface of the axial bore 720.

Once the constrained variable volume 744 has been completely contracted, the process may then be repeated, either another cycle for the first fluid reservoir 726 or a dispense cycle for the second fluid reservoir 727. A dispense cycle for the second fluid reservoir 727 as shown in FIGS. 11E-11H may be initiated by translating the constrained variable volume 744 to a position in fluid communication with the second inlet port 739 as shown in FIG. 11E. A translational axial force in a proximal direction may then be exerted through a boundary section of the constrained variable volume 744 (as discussed above) to expand the constrained variable volume 744 and draw fluid into the constrained variable through the second inlet port 739 from the second fluid reservoir 727 as shown in FIG. 11F. The constrained variable volume 744 may then be translated by the drive mechanism 750 to a position in fluid communication with the outlet port 742 as shown in FIG. 11G by the application of additional axial translation force in a proximal direction. Translational axial force in a distal direction may then be exerted through a boundary section of the constrained variable volume 744 so as to at least partially contract the constrained variable volume 744 and dispense fluid from the constrained variable volume 744 through the outlet port 742 to a patient. As discussed above, the maximum dispense pressure may be determined at this step by the maximum distal axial force that may be generated by the drive mechanism 750 as well as the frictional force between the seal or seals of the distal spool section 757 and the inner surface of the axial bore 720. With regard to these dispense methods, in some cases, the first fluid reservoir 726 and second fluid reservoir 727 may contain the same fluid which may be a therapeutic agent. In some cases, the first fluid reservoir 726 contains a first therapeutic agent for delivery to a patient and the second fluid reservoir 727 contains a second therapeutic agent different from the first therapeutic agent for delivery to a patient. As such, a delivery method includes delivering a first therapeutic agent and a second therapeutic agent different from the first therapeutic agent to a patient. In some circumstances, the first therapeutic agent may include a fast acting insulin compound and the second therapeutic agent may include a slow acting insulin compound.

The dispense cycle for dispensing fluid from either the first fluid reservoir 726 or the second fluid reservoir 727 may be repeated independently as for as many cycles as desired. Thus, the configuration shown allows fluid to be dispensed from the first fluid reservoir 726 independently of dispensing fluid from the second fluid reservoir 727, and vice versa. It should be noted, however, that some mixing of fluid from the first fluid reservoir 726 and second fluid reservoir 727 will naturally occur during the dispense cycle as the constrained variable volume 744 of this embodiment passes over both inlet ports 738 and 739 during normal operation. In addition, the constrained variable volume 744 does not typically completely empty at the conclusion of a dispense cycle.

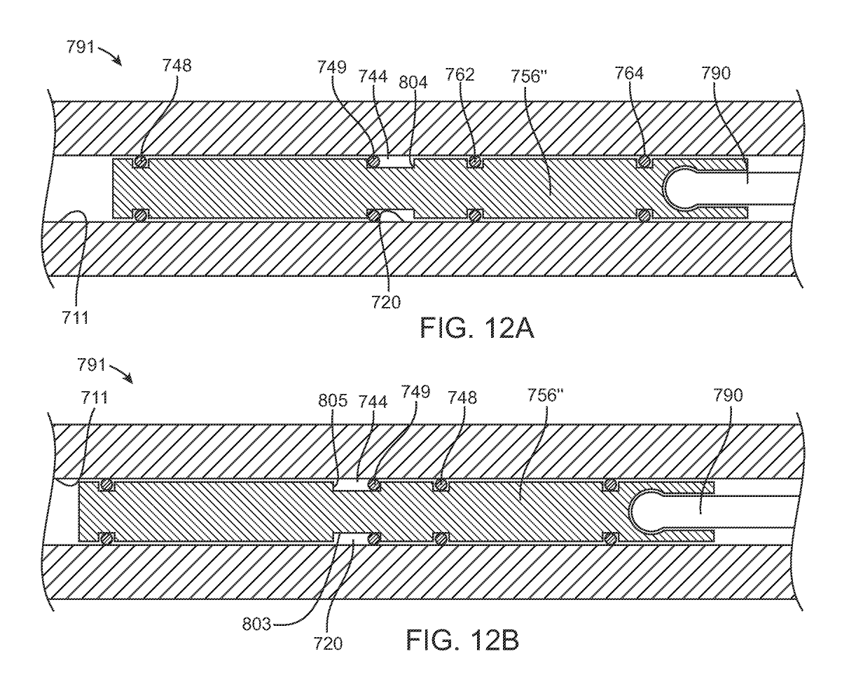

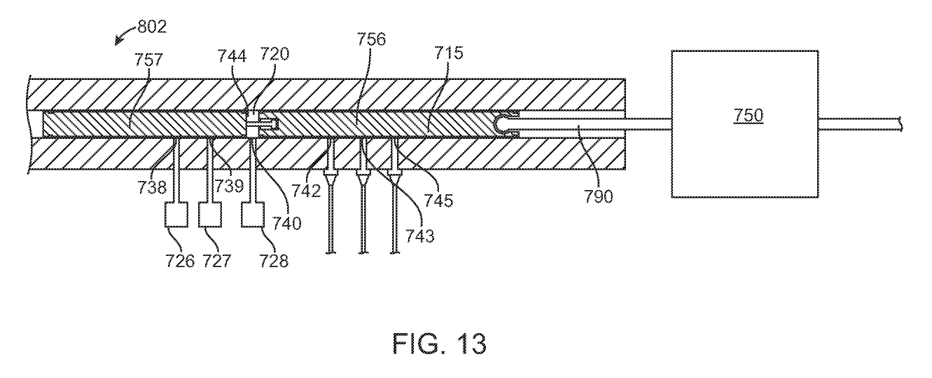

Although the embodiment of FIG. 11AA includes two fluid reservoirs 726 and 727 and a single outlet port 742, any desired combination or number of inlet ports and outlet ports may be used for the spool 756 configuration shown, so long as the spacing and locations of the ports are appropriate. For example, FIG. 13 illustrates a delivery mechanism embodiment 802 with three fluid reservoirs 726, 727 and 728 and three respective inlet ports 738, 739, and 740. The embodiment also includes three outlet ports 742, 743, and 745. A dispense cycle for such an embodiment may also include translating the constrained variable volume 744 to a position in fluid communication with a third inlet port 740, exerting translational force through a boundary section of the constrained variable volume 744 to expand the constrained variable volume 744 and draw fluid into the constrained variable through the third inlet port 740 from a third reservoir 728 and translating the constrained variable volume 744 to a position in fluid communication with an outlet port 742, 743 or 745. Thereafter, translational axial force may be exerted in a distal direction through a boundary section of the constrained variable volume 744 so as to at least partially contract the constrained variable volume 744 and dispense fluid from the constrained variable volume 744 through an outlet port to a patient.

In addition, although the constrained variable volume 744 of the embodiment of FIG. 11AA is formed between two spool sections 715 and 757 which are axially displaceable relative to each other, other embodiments may be used. For example, FIGS. 12A and 12B illustrate a delivery mechanism embodiment 791 having a spool embodiment 756″ that includes a constrained variable volume 744 formed by a first fixed seal 762 and a second seal 749 which is disposed in a sliding configuration relative to the spool body 756″ but with limited axial displacement. More specifically, the spool 756″ embodiment of FIGS. 12A and 12B is configured to form a constrained variable volume 744 in conjunction with the inner surface 711 of the axial bore 720 the first seal 762 disposed between and in sealed relation with the spool 756″ and the bore 720, a second seal 749 disposed between and in sealed relation with the spool 756″ and the bore 720 and an outside surface of the spool 756″ disposed between the first and second seal 762 and 749. For the embodiment shown, the first seal 762 lies in a gland of the spool 756″ so as to be substantially axially fixed relative to the spool 756″ but displaceable relative to an inside surface 711 of the bore 720. The second seal 749 which is disposed distally of the first seal 762 lies in an axially extended gland or slide portion 803 of the spool 756″. The second seal 749 is disposed between and in sealed relation with an outside surface of the slide portion 803 of the spool 756″ and an inside surface 711 of the bore 720. The second seal 749 is configured to slide over the slide portion 803 of the spool 756″, which forms a substantially fluid tight but displaceable seal between an outside surface of the slide portion 803. The second seal 749 is also configured to slide over the inside surface 711 of the bore 720. However, in order to operate properly, the friction force between an outer surface of the second seal 749 and inner surface 711 of the axial bore 720 may be greater than the frictional resistance to sliding of the inner surface of the second seal 749 against the slide portion 803 of the spool 756″. Otherwise, the second seal 749 would remain essentially axially fixed relative to the spool 756″ and the volume between the first and second seals 762 and 749 would not vary substantially in order to provide the drawing and dispensing action as discussed above with regard to FIGS. 11A-11G.

For such a spool 756″ embodiment, a shaft of the spool 756″ may be axially continuous and rigid along an axial direction and the drive mechanism may be directly coupled to the rigid shaft 790 and constrained variable volume 744 formed by the shaft 756″ and seals 749 and 762 disposed thereon. In some cases, the slide portion 803 of the spool 756″ may include a smooth reduced diameter axial section of a shaft of the spool 756″. The slide portion 803 may have a substantially continuous transverse cross section, a first stop 804 at an end of the slide portion 803 which is configured to limit axial movement of the second seal 749 over the slide portion 803 and a second stop 805 opposite the slide portion 803 of the first stop 804 that is also configured to limit axial movement of the second seal 749 over the slide portion 803. The maximum and minimum volume of the constrained variable volume 744 formed by such a sliding seal spool may be determined at least in part by the separation of the first stop 804 and second stop 805 and the axial thickness of the second seal 749 disposed on the slide portion 803 between the stops 804 and 805. FIG. 12A shows the second seal 749 in a distal most position which would correspond to a maximum volume configuration for the constrained variable volume 744 formed by the seals. FIG. 12B shows the second seal 749 in a proximal most position which would correspond to a minimum volume configuration for the constrained variable volume 744 formed by the seals.

For any of the infusion pump embodiments discussed herein, it may be desirable to include a cartridge configuration with a disposable portion or component and reusable portion or component. In some cases, the disposable portion may generally include the delivery mechanism 132, 714 etc. and the fluid reservoirs, such as the first fluid reservoir 726 and second fluid reservoir 727. In some cases, the reusable component may include the drive mechanism 750 which may have a drive shaft 790 which may be detachably coupled to the spool 756 by a ball 794 and socket arrangement or any other suitable arrangement. The reusable component may also include the controller 768, a power storage cell such as a battery (not shown) or the like.

As discussed above with regard to the infusion pump embodiment of FIG. 11A, some mixing of the fluids being delivered from the first fluid reservoir 726 and the second fluid reservoir 727 may occur during the dispense cycle of the delivery mechanism 714. This arrangement may be suitable for some applications, but in some cases, it may be desirable to dispense two separate fluids from two separate fluid reservoirs with a single delivery mechanism 714 and drive mechanism 750 without any mixing of the two fluids within the delivery mechanism. In some cases, the two or more fluids being delivered may still be mixed once they have been dispensed from a constrained variable volume 744 of the delivery mechanism, but not while being moved through the inlet ports 738 and 739 and axial bore 720 of the delivery mechanism. For example, in embodiments that include two or more fluid reservoirs 726 and 727 and inlet ports 738 and 739 but only one outlet 742 or dispense port, the two or more multiple fluids may be mixed in the outlet port 742 and any tubing extending from the outlet tube to a patient or other intended destination for the fluids. In some cases, it may further be desirable to include multiple outlet ports, in some cases, at least one outlet port 742 for each distinct fluid being delivered or dispensed from the infusion pump.

FIG. 14A illustrates a delivery mechanism 806 that is configured to deliver two separate fluids from two separate fluid reservoirs to two separate respective outlet ports without mixing the two fluids. In some cases, two outlet ports 904 and 906 may be combined into a single conduit where the fluids would be mixed if such a single outlet tube was desired. As discussed above, the use of a single spool and drive mechanism 750 may be useful with regard to cost of the device, an efficient use of stored energy or battery life and overall size and weight of the device. The drive mechanism 750 used in some cases for the delivery mechanism 806 embodiment of FIG. 14A may be the same as or similar to the drive mechanism 750 of the infusion pump system 110 shown in FIGS. 6-10 and discussed above. As such, the proximal end of the spool 902 of the delivery mechanism 806 in FIG. 14A may include a coupling, such as a ball coupling 794, that is suitable for releasable connection to a distal end of a drive shaft such as drive shaft 790 shown in the embodiment of FIGS. 6-10. The delivery mechanism 806 of FIG. 14A may also have any other features, dimensions, modes of operation or materials which are the same as or similar to those of the delivery mechanism embodiment 110 shown in FIGS. 6-10. In some cases, the spool 902 may actuated by a drive mechanism 750 including a motor powered by a power storage cell and controlled by a controller 768.

In general, an infusion pump embodiment as shown in FIG. 14A may include a first inlet port 903, a first outlet port 904, a first fluid reservoir 919 in fluid communication with the first inlet port 903, a second inlet port 905, a second outlet port 906 and a second fluid reservoir 917 in fluid communication with the second inlet port 905. A first constrained variable volume 935 may be translatable between a position in fluid communication with the first inlet port 903 and a position in fluid communication with the first outlet port 904. A second constrained variable volume 937 may be translatable between a position in fluid communication with the second inlet port 905 and a position in fluid communication with the second outlet port 906. In some cases, the drive mechanism 750 may be configured to expand or contract the first constrained variable volume 935 and second constrained variable volume 937 due to exertion of a translational force through a boundary section of the respective constrained variable volumes 935 and 937.

More specifically, the delivery mechanism embodiment 806 shown in FIG. 14A include an axial bore 900. The axial bore 900 may have a first axial bore section 901, a second axial bore section 940 and a spool 902 disposed in the axial bore 900. The first axial bore section 901 may include the first inlet port 903 and the first outlet port 904 with said first ports being in fluid communication with an interior volume of the first axial bore section 901. The second axial bore section 940 may include the second inlet port 905 and second outlet port 906 with said second ports 905 and 906 being in fluid communication with an interior volume of the second axial bore section 940.

The spool 902 may be axially translatable within the axial bore 900 and have a first spool section 907 including a proximal end 907′ configured to couple to a drive shaft 790 of a drive mechanism such as the drive mechanism 750 shown in FIGS. 6-10 above. The first spool section 907 also has a first seal 908 which forms a fluid tight seal between the first spool section 907 and an interior surface of the axial bore 900. The first seal 908 or elements thereof may be disposed in a gland or glands of the first spool section 907 in some cases so as to be substantially axially fixed relative to the first spool section 907. The first seal 908 of the spool 907 embodiment shown includes a double element seal that includes two o-ring type seal elements 909 that are axially spaced from each other. The double seal elements of the first seal 908 may provide stability for the first spool section 907 as well as a slidable seal arrangement with respect to the axial bore. An outer surface of the first seal 908 may be slidable relative to the interior surface of the axial bore 900 to allow the first section of the spool 907 to move axially within the axial bore 900 while maintaining a fluid tight seal therewith.

The spool 902 may also include a second spool section 910 having a proximal end coupled to a distal end of the first spool section 907 by a limited displacement coupling. The second spool section 910 may include a second seal 911 which forms a fluid tight seal between the second spool section 910 and the interior surface of the axial bore 900. The second seal 911 may also be substantially axially fixed relative to the second spool section 910 and slidable relative to the interior surface of the axial bore 900 as with the first seal 908. The second seal 911 also includes a double seal arrangement with a double seal element that includes two o-ring type seals 909 which are axially spaced from each other. The spool 902 may also have a third spool section 914 which has a proximal end coupled to a distal end of the second spool section 910 by a limited displacement coupling. The third spool section 914 may also have a third seal 915 which forms a fluid tight seal between the third spool section 914 and the interior surface of the axial bore 900. The third seal 915 may be substantially is axially fixed relative to the third spool section 914 and axially slidable relative to the interior surface of the axial bore 900 as with the first seal 908. The third seal 915 also includes a double seal arrangement with a double seal element that includes two o-ring type seals 909 which are axially spaced from each other.

The first constrained variable volume 935 may be formed between the first spool section 907, the second spool section 910, the first seal 908, the second seal 911 and the interior surface of the axial bore 900. In such a configuration, the first and second seals 908 and 911 are axially translatable relative to each other but mechanically constrained to a maximum and minimum axial separation over a limited axial distance by the limited displacement coupling operatively coupled between the first spool section 907 and second spool section 910. The second constrained variable volume 937 may be formed between the second spool section 910, the third spool section 914, the second seal 911, the third seal 915 and the interior surface of the axial bore 900. With this configuration, the second and third seals 911 and 915 are axially translatable relative to each other but mechanically constrained to a maximum and minimum axial separation over a limited axial distance by the limited displacement coupling operatively coupled between the second spool section 910 and third spool section 914.

The infusion pump includes the first fluid reservoir 919 having an interior volume 928 in fluid communication with the first inlet port 903 and the second fluid reservoir 917 having an interior volume 929 in fluid communication with the second inlet port 905. The first fluid reservoir 919 and second fluid reservoir 917 embodiments shown in FIG. 14A include collapsible reservoirs bounded by a thin flexible fluid tight material 921. This configuration may allow the contents of the reservoirs 917 and 919 to be withdrawn without the need to vent the interior volume 928 of the reservoir itself. However, in some cases, a substantially rigid fluid tight shell 922 may be disposed about the first and second fluid reservoirs 917 and 919 with a fluid tight interior volume 930 being formed between an inside surface of the rigid shell 922 and respective outside surfaces of the first and second fluid reservoirs 917 and 919. In some circumstances, the interior volume 930 of the rigid shell 922 forms a vented volume 930 which allows the volume between the outer surfaces of the collapsible reservoirs 917 and 919 and inner surface of the rigid shell 922 to accommodate changes in volume of the reservoirs as fluid is withdrawn. For such embodiments, a vent inlet port 924 may be disposed in fluid communication with the interior volume of the bore 900 and an interior volume of the vented volume 930. A vent outlet port 925 may be in fluid communication between an interior volume 930 of the bore 900 and the ambient atmosphere. The spool 902 may include a pair of seals to form a vent volume 923 such that the vented volume 930 of the substantially rigid shell 922 may be vented through a conduit 931 in communication between the vented volume 930 and axial bore 900 and out to the ambient atmosphere through the vent outlet port 925.

As discussed above, the fluid reservoirs 917 and 919 may be disposed in a substantially rigid shell 922 having an interior volume 930 that is a vented volume. When venting of the vented volume 930 is desirable, the controller 768 may be configured to deliver a drive signal to the drive mechanism 750 so as to axially translate the spool 902 to a position whereby the vent volume 930 of the spool 902 is in fluid communication with the vent inlet port 924 and vent outlet port 925. In this position, the vented volume 930 is put into fluid communication with the ambient atmosphere or any other desirable or predetermined environment. In addition, a pressure sensor 932 may be disposed within the vented volume 930 or in fluid communication with the vented volume 930 to determine the pressure within the vented volume 930. Such pressure measurements may be useful for determining the amount of fluid dispensed from the fluid reservoirs 917 and 919, determining when venting of the vented volume 930 is necessary, detecting leaks or clogs in the infusion pump system or the like.

In some instances, the drive mechanism 750 may be operatively coupled to the proximal end of the first spool section 907. In some such embodiments, the drive mechanism 750 may be configured to axially translate the first constrained variable volume 935 between the first inlet port 903 and first outlet port 904 and configured to expand the first constrained variable volume 935 while in fluid communication with the first inlet port 903 and contract the first constrained variable volume 935 while in fluid communication with the first outlet port 904. The drive mechanism 750 may also be configured to axially translate the second variable volume 937 between the second inlet port 905 and second outlet port 906 and configured to expand the second constrained variable volume 937 while in fluid communication with the second inlet port 905 and contract the second constrained variable volume 937 while in fluid communication with the second outlet port 906.

For the constrained variable volume embodiments 935 and 937 shown, the constrained variable volumes 935 and 937 may be expanded or contracted by exertion of axial force through a boundary section of each respective constrained variable volume. In particular, when the drive mechanism 750 imparts axial force on the proximal section of the first spool section 907, that force may be exerted against the contents of the first constrained variable volume 935 by the distal end of the first spool section 907 and first seal 908. The distal end of the first spool section 907 and first seal 908 on the first spool section 907 form a boundary section of the constrained variable volume 935 and axial force from the drive mechanism 750 may be applied to the contents of the constrained variable volume 935 in this way. Resulting axial force may thus be applied to a proximal portion of the second spool section 910 by increased pressure of the contents of the first constrained variable volume 935 pushing against the proximal end of the second spool section 910 and second seal 911, which also may be considered to form a boundary section of the first constrained variable volume 935. If the first constrained variable volume 935 is in fluid communication with the first inlet port 903 or first outlet port 904, the increased pressure or decreased pressure resulting from axial force being applied to the first spool section 907 by the drive mechanism 750 may cause the first constrained variable volume 935 to expand or contract. Such an action may thus draw fluid into the expanding first constrained variable volume 935 or deliver fluid from a contracting first constrained variable volume 935. The same result occurs for the second constrained variable volume 937, except that the axial force applied to the second constrained variable volume 937 is transmitted by the second spool section 910 to the third spool section 914 through a boundary section of the second constrained variable volume 937. In the case of the second constrained variable volume 937, the boundary section may include the distal end of the second spool section 910 and second seal 911 on the second spool section 910.

In some cases, the drive mechanism 750 and spool 902 may be configured to translate the spool 902 without the first constrained variable volume 935 overlapping the second axial bore section 940 or the second constrained variable volume 937 overlapping the first axial bore section 901. This type of arrangement may be useful in preventing mixing of fluids which are being dispensed from the respective fluid reservoirs. In some instances, the first axial bore section 901 may include a plurality of first inlet ports 903 in fluid communication with the first fluid reservoir 919. The delivery mechanism 806 may also be configured such that the first constrained variable volume 935 is positionable to overlap all of the plurality of first inlet ports 903 of the first fluid reservoir 919 independent of an overlap with an inlet port in fluid communication with another fluid reservoir. Likewise, the second axial bore section 940 may include a plurality of second inlet ports 905 in fluid communication with the second fluid reservoir 917 and the delivery mechanism 806 may be configured such that the second constrained variable volume 937 is positionable to overlap all of the plurality of second inlet ports 905 of the second fluid reservoir 917 independent of an overlap with an inlet port in fluid communication with another fluid reservoir. In some cases, the infusion pump may further include a proximal bore seal and a distal bore seal disposed between an outside surface of the spool 902 and an inside surface of the bore 900 at respective proximal and distal ends of the spool. The bore seals may be configured to isolate the inlet and outlet ports 903, 904, 905 or 906 of the bore 900 from the surrounding environment.

FIGS. 14A-16F illustrate delivery sequence and venting sequence embodiments. A delivery sequence embodiment may be initiated in some cases by first filling the second constrained variable volume 937 from the second fluid reservoir 917 through second inlet port 905. With the second constrained variable volume 937 in fluid communication with the second inlet port 905, the drive mechanism 750 may be actuated to apply axial tension on the first spool section 907 which is then transmitted to the second spool section 910 as shown by the arrow 920 in FIG. 14A. FIG. 14B shows the second constrained variable volume 937 completely filled with fluid from the second fluid reservoir 917. During the fill step, the third spool section 914 remains substantially stationary and a second limited displacement coupling 946 is axially expanded so as to expand the second constrained variable volume 937. Fluid is drawn into the second constrained variable volume as indicated by arrow 939 in FIG. 14B. As shown, an enlarged portion 942 of the extension 943 of the second limited displacement coupling 946 has been displaced to a position adjacent a shoulder portion of a cavity 944 of the second limited displacement coupling 946. It should be noted that a first limited displacement coupling 945 has remained in an axially contracted configuration during the fill process of the second constrained variable volume 937 because the first constrained variable volume 935 between the first spool section 907 and second spool section 910 is not in fluid communication with any inlet or outlet port and the volume thus remains hydraulically locked together.

Once the second constrained variable volume 937 has been filled, the third spool section 914 is further proximally retracted or displaced until the first constrained variable volume 935 comes into fluid communication with the first inlet port 903 as shown in FIG. 14C. Once the first constrained variable volume 935 is in fluid communication with the first inlet port 903, the negative pressure caused by the axial friction and drag of the seals of the second spool section 910 and first spool section 907 causes the first constrained variable volume 935 to begin to fill as shown by the arrow 926 in FIG. 14C. The first constrained variable volume 935 continues to fill with fluid from the first fluid reservoir 919 through the first inlet port 903 until the enlarged portion 944′ of the extension 947 of the first limited displacement coupling 945 is disposed adjacent a shoulder portion of the cavity 948 of the first limited displacement coupling 945 as shown in FIG. 14D. At this stage, all three spool sections 907, 910 and 914 are mechanically coupled with both limited displacement couplings 945 and 946 fully extended as shown in FIG. 14D.

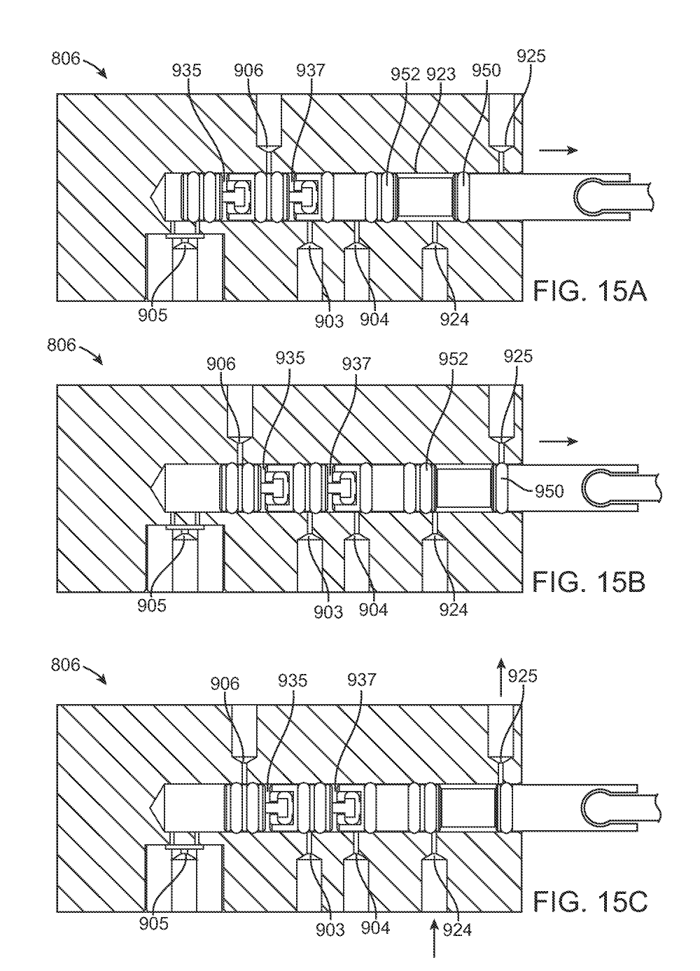

With both constrained variable volumes 935 and 937 completely filled, a venting cycle is initiated by further axially displacing the spool 902 in a proximal direction as shown in FIG. 15A. Proximal retraction or displacement of the spool 902 continues as shown in FIG. 15B with the vent volume 923 of the spool 902 formed between a first vent seal 950 and a second vent seal 952 disposed adjacent the vent inlet port 924 and vent outlet port 925. As the spool 902 is further translated in a proximal direction, the vent volume 923 formed between the first vent seal 950 and second vent seal 952 becomes disposed in fluid communication with both the vent inlet port 924 and vent outlet port 925 so as to create a vent conduit extending from the vent inlet port 924 to the ambient atmosphere outside the vent outlet port 925 as shown in FIG. 15C. Air or any other fluid disposed within the vented volume 930 may be vented to the atmosphere as shown by the arrows in FIG. 15C. Air or any other fluid from the ambient atmosphere (or any other volume in fluid communication with the vent outlet port 925 may also be vented inward into the vented volume 930 in a direction opposite that of the vent arrows shown in FIG. 15C. Such a venting process would be typical as fluid is withdrawn from the first and second fluid reservoirs 917 and 919, air from the ambient atmosphere may be drawn into the vented volume 930 in order to fill the void left in the vented volume 930 by the dispensed fluid.

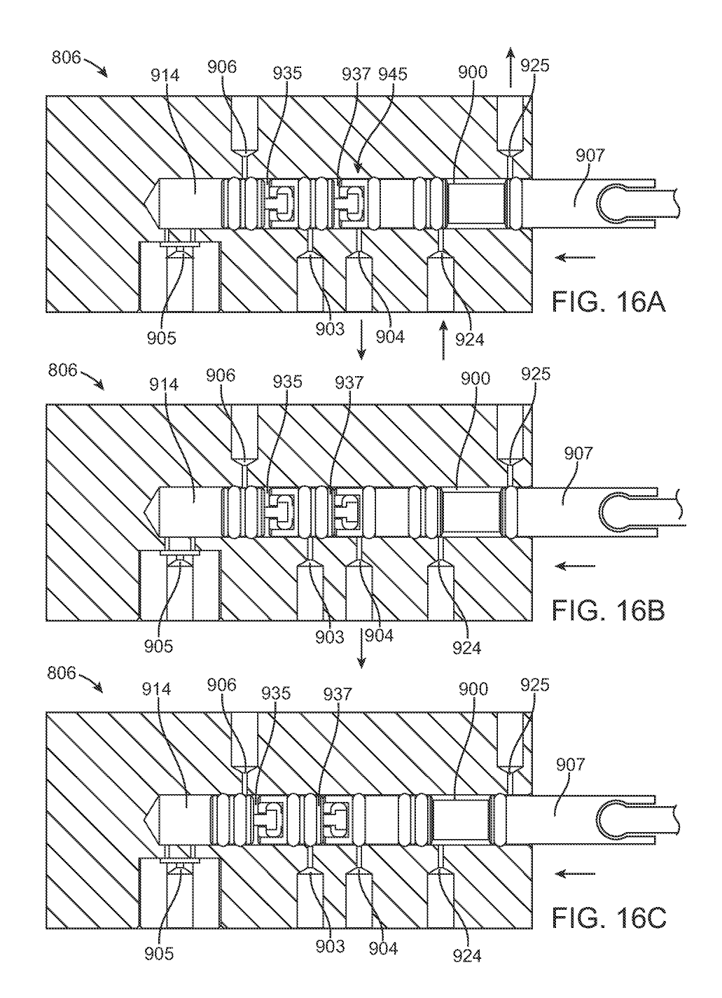

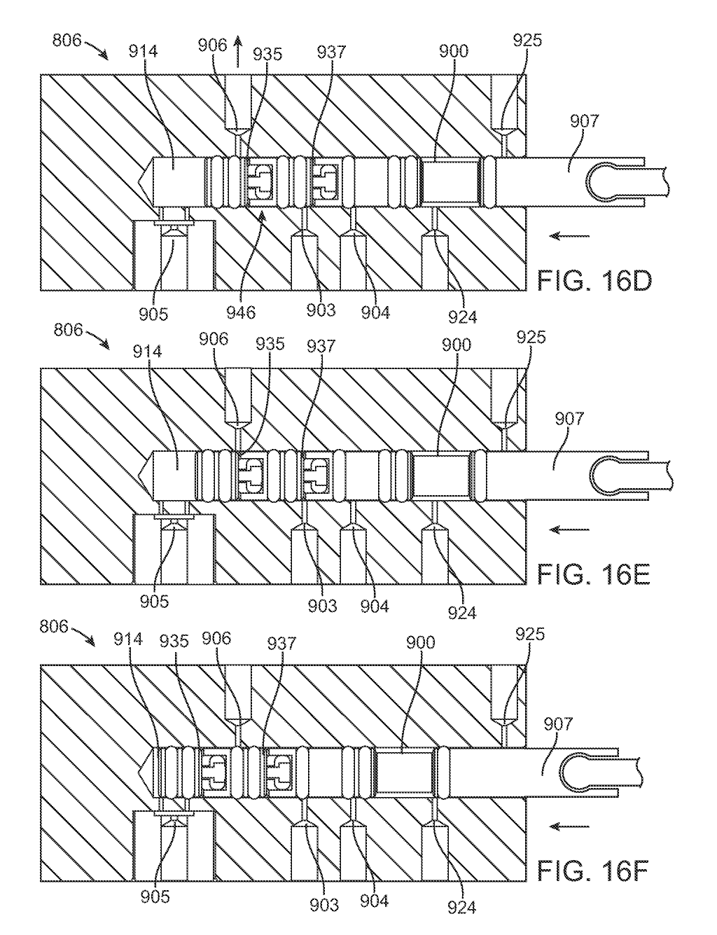

Once the vented volume 930 has been vented, or at any other suitable time prior to venting, the drive mechanism 950 may begin to apply a distal axial force to the spool 902, and, in particular, to the proximal end of the first spool section 907 as shown in FIG. 16A. As the axial force from the drive mechanism 950 is exerted onto the first spool section 907, each of the first and second constrained variable volumes 935 and 937 are compressed due to the friction of the seals of the second spool section 910 and third spool section 914 which increases the pressure of the fluid in the filled volumes. Because the first constrained variable volume 935 is in fluid communication with the first outlet port 904 at this stage, the increased pressure within the first constrained variable volume 935 delivers the fluid from the first constrained variable volume 935 from the first outlet port 904 as shown by the arrows in FIGS. 16 A and 16B. The delivery process of fluid from the first volume 935 continues until the first limited displacement coupling 945 bottoms out and the first spool section 907 comes into solid mechanical contact with the second spool section 910 as shown in FIG. 16B. As distal axial force is further applied, the spool 902 assembly begins to advance together in a distal direction until the second constrained variable volume 937 comes into fluid communication with the second outlet port 906 as shown in FIG. 16C. Once the second constrained variable volume 937 is in fluid communication with the second outlet port 906, the fluid in the second volume 937 begins to be dispensed or delivered from the second constrained variable volume 937 as shown by the arrow in 16D. The delivery of fluid from the second constrained variable volume 937 continues through the second outlet port 906 until the second limited displacement coupling 946 bottoms out and the third spool section 914 once again begins to advance in a distal direction as shown in 16E. The distal advancement of the spool 902 continues until the spool 902 is returned to the home position as shown in FIG. 16F, at which point, the delivery and vent cycle may begin again.

It should be noted that while fluid was delivered from both the first fluid reservoir 919 and second fluid reservoir 917 for the delivery cycles discussed above, fluid may also be delivered from each of the fluid reservoirs 917 and 919 repeatedly and independently of delivery of fluid from the other fluid reservoir. For example, the second constrained variable volume 937 may be repeatedly shuttled back and forth from a position in fluid communication with the second inlet port 905 to a position in fluid communication with the second outlet port 906 without ever making a fluid delivery to the first outlet port 904. The second volume 937 also never overlaps the first section 901 of the axial bore 900 and vice versa. As such, there is no cross-contamination between the fluid within the first fluid reservoir 919 and the fluid within the second fluid reservoir 917. As discussed above, in some cases the first fluid reservoir 919 may contain a first therapeutic agent for delivery to a patient and the second fluid reservoir 917 may contain a second therapeutic agent for delivery to a patient. In addition, in some cases, the first therapeutic agent may include a fast acting insulin compound and the second therapeutic agent may include a slow acting insulin compound.

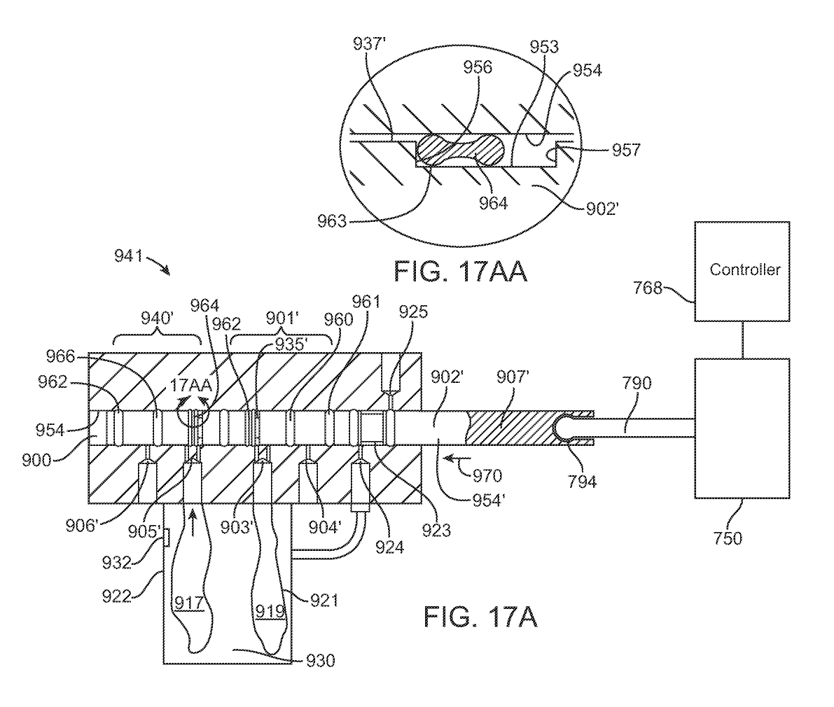

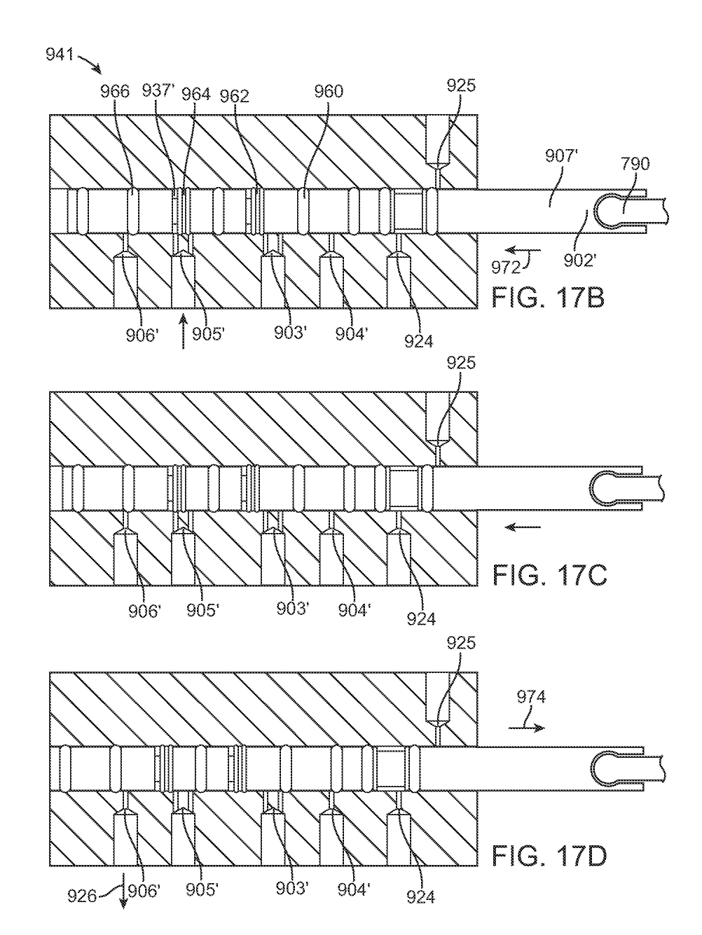

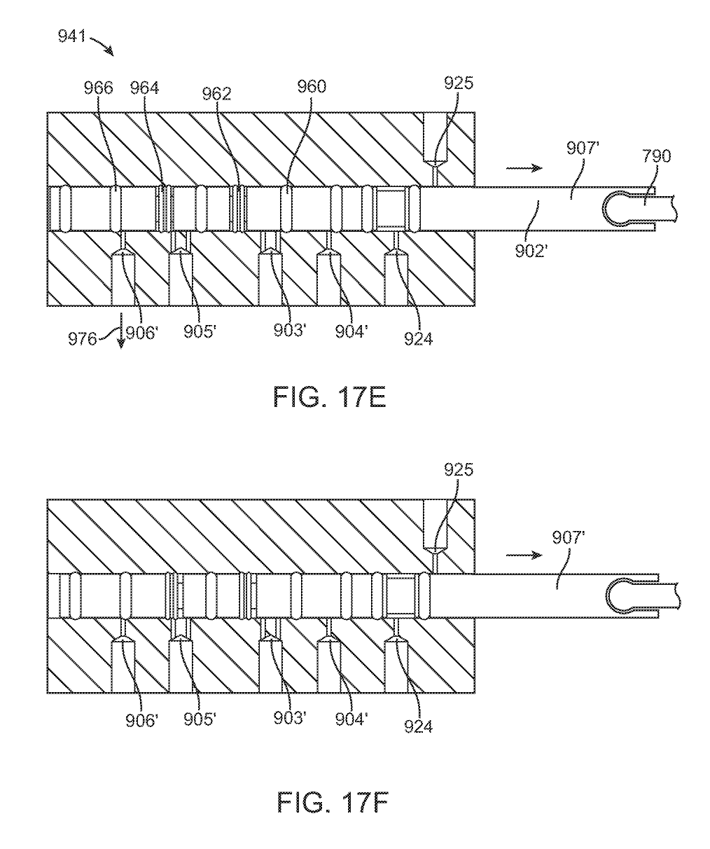

Another embodiment of a delivery mechanism 941 that may be used to deliver two fluids from two different fluid reservoirs 917 and 919 with a single drive mechanism 750 is shown in FIG. 17A. This embodiment may also be used to deliver to different fluids from two different fluid reservoirs 917 and 919 without mixing of the fluids within the delivery mechanism 941. In some cases, the two outlet ports 904′ and 906′ may be combined into a single conduit where the fluids would be mixed if such a single outlet tube was desired. As discussed above, the use of a single spool 902′ and drive mechanism 750 may be useful with regard to cost of the device, an efficient use of stored energy or battery life and overall size and weight of the device. The drive mechanism 750 used in some cases for the delivery mechanism 941 embodiment of FIG. 17A may be the same as or similar to the drive mechanism 756 of the infusion pump system 110 shown in FIGS. 6-10 and discussed above. As such, the proximal end of the spool 902′ of the delivery mechanism 941 in FIG. 17A may include a coupling, such as a ball coupling 947, that is suitable for releasable connection to a distal end of a drive shaft such as drive shaft 790 shown in the embodiment of FIGS. 6-10. The delivery mechanism 941 of FIG. 17A may also have any other features, dimensions, modes of operation or materials which are the same as or similar to those of the delivery mechanism 714 embodiment shown in FIGS. 6-10. In some cases, the spool 902′ may actuated by a drive mechanism 750 including a motor powered by a power storage cell (not shown) and controlled by a controller 768.

The delivery mechanism 941 embodiment shown in FIG. 17A includes two constrained variable volumes 935′ and 937′, each formed by an axially fixed seal and another seal which is axially spaced from the fixed seal and disposed in a sliding configuration relative to the spool 902′ body but over a limited axial displacement. More specifically, the spool embodiment 902′ of FIG. 17A is configured to form at least two constrained variable volumes 935′ and 937′ in conjunction with the inner surface 954 of an axial bore 900′. The constrained variable volumes 935′ and 937′ may be bounded by an inside surface of the bore 900′, an axially fixed seal disposed between and in sealed relation with the spool 902′ and the bore 900′, a sliding seal disposed between and in sealed relation with the spool 902′ and the bore 900′ and an outside surface of the spool 902′ disposed between the fixed and sliding seals.

For the embodiment shown, the fixed seal may be disposed in a circumferential groove or gland of the spool 902′ so as to be substantially axially fixed relative to the spool 902′ but displaceable relative to the inside surface 954 of the bore 900′. The sliding seals may lie in an axially extended gland or groove of the spool 902′ which may be referred to as a slide portion of the spool 902′. The sliding seals 951 may be disposed between and in sealed relation with an outside surface of the slide portion of the spool 902′ and an inside surface of the bore 900′. The sliding seals 951 may be configured to slide over the slide portion of the spool 902′, which forms a substantially fluid tight but displaceable seal between an outside surface of the slide portion. The sliding seals may also be configured to slide over the inside surface of the bore 900′. However, in order to operate properly, the friction force between an outer surface of the sliding seal and inner surface 954 of the axial bore 900′ may be greater than the frictional resistance to sliding of the inner surface 954 of the sliding seal against the slide portion of the spool 902′. Otherwise, the sliding seal would remain essentially axially fixed relative to the spool 902′ and the volume between the fixed and sliding seals and may not vary substantially.

For such a spool embodiment 902′, a shaft 954′ of the spool 902′ may be axially continuous and rigid along an axial direction and the drive mechanism 750 may be directly coupled to the rigid shaft 954′ and constrained variable volume 935′ formed by the shaft and annular seals disposed thereon. In some cases, the slide portions of the spool 902′ may include a smooth reduced diameter axial section of a shaft 954′ of the spool 902′. The slide portions may have a substantially continuous transverse cross section, a first stop at an end of the slide portion which is configured to limit axial movement of the sliding seals over the slide portions and a second stop opposite the slide portion of the first stop that is also configured to limit axial movement of the sliding seals over the slide portion. The maximum and minimum volume of the constrained variable volume 935′ and 937′ formed by such a sliding seal spool may be determined at least in part by the separation of the first stop and second stop of each variable volume and the axial thickness of the sliding seals disposed on the slide portion between the stops.

Some embodiments of an infusion pump may include a delivery mechanism 941 having an axial bore 900′ with a first axial bore section 901′ and a second axial bore section 940′. The first axial bore section 901′ may include a first inlet port 903′ and a first outlet port 904′ with said first inlet and first outlet ports 903′ and 904′ being in fluid communication with an interior volume of the first axial bore section 901′. The second axial bore 940′ section may be axially spaced from the first axial bore section 901′ and may include a second inlet port 950′ and second outlet port 906′ with said second inlet and outlet ports 905′ and 906′ being in fluid communication with an interior volume of the second axial bore section 940′. The delivery mechanism 941 may also include the spool 902′ which includes a proximal end and a distal end, which is disposed within the axial bore 900′, which is axially translatable within the axial bore 900′, which forms the first constrained variable volume 935′ between a first seal 960 axially fixed relative to the spool 902′, a slidable second seal 962 disposed distally of the first seal 960, and an interior surface 954 of the axial bore 900′. The second constrained variable volume 937′ may be formed between a third slidable seal 964 disposed distally of the second slidable seal 962, a fourth seal 966 disposed distally of the third slidable seal 964 and axially fixed relative to the spool 902′ and an interior surface 954 of the axial bore 900′. In some cases, a center seal 968 which is substantially axially fixed with respect to the spool 902′ may be disposed between the second slidable seal 962 and the third slidable seal 964. An enlarged view of the third slidable seal 964 having an outer surface disposed in contact and sealed relation with an interior or inner surface 954 of the axial bore 900′ and outer surface of the slide portion 953 of the spool 902′ is shown in FIG. 17AA.

Some infusion pump embodiments may include the first fluid reservoir 919 having an interior volume in fluid communication with the first inlet port 903′, the second fluid reservoir 917 having an interior volume in fluid communication with the second inlet port 905′ and the drive mechanism 750 which is operatively coupled to the proximal end of the spool 902′. In some cases, the drive mechanism 750 may be configured to axially translate the first constrained variable volume 935′ between the first inlet port 903′ and first outlet port 904′ and configured to expand the first constrained variable volume 935′ while in fluid communication with the first inlet port 903′ and contract the first constrained variable volume 935′ while in fluid communication with the first outlet port 904′. The drive mechanism 750 may also be configured to axially translate the second variable volume 937′ between the second inlet port 905′ and second outlet port 906′ and configured to expand the second constrained variable volume 937′ while in fluid communication with the second inlet port 905′ and contract the second constrained variable volume 937′ while in fluid communication with the second outlet port 906′. For some embodiments, the first fluid reservoir 919 contains a first therapeutic agent for delivery to a patient and the second fluid reservoir 917 contains a second therapeutic agent different from the first therapeutic agent for delivery to a patient. As such, a delivery method includes delivering a first therapeutic agent and a second therapeutic agent different from the first therapeutic agent to a patient. In some circumstances, the first therapeutic agent may include a fast acting insulin compound and the second therapeutic agent may include a slow acting insulin compound.

The first fluid reservoir 919 and second fluid reservoir 917 embodiments shown in FIG. 17A include collapsible reservoirs bounded by the thin flexible fluid tight material 921. This configuration may allow the contents of the reservoirs to be withdrawn without the need to vent the interior volume of the reservoir itself. However, in some cases, the substantially rigid fluid tight shell 922 may be disposed about the first and second fluid reservoirs 917 and 919 with a fluid tight interior volume 930 being formed between an inside surface of the rigid shell 922 and respective outside surfaces of the first and second fluid reservoirs 917 and 919. In some circumstances, the interior volume 930 of the rigid shell 922 forms a vented volume 930 which allows the volume between the outer surfaces of the collapsible reservoirs 917 and 919 and inner surface of the rigid shell 922 to accommodate changes in volume of the reservoirs as fluid is withdrawn. For such embodiments, a vent inlet port 924 may be disposed in fluid communication with the interior volume of the bore 900′ and an interior volume of the vented volume 930. A vent outlet port 925 may be in fluid communication between an interior volume of the bore 900′ and the ambient atmosphere. The spool 902′ may include a pair of seals to form a vent volume such that the vented volume 930 of the substantially rigid shell 922 may be vented through a conduit in communication between the vented volume 930 and axial bore 900′ and out to the ambient atmosphere through the vent outlet port 925.

When venting of the vented volume 930 is desirable, the controller 768 may be configured to deliver a drive signal to the drive mechanism 750 so as to axially translate the spool 902′ to a position whereby the vent volume 923 of the spool 902′ is in fluid communication with the vent inlet port 924 and vent outlet port 925. In this position, the vented volume 930 is put into fluid communication with the ambient atmosphere or any other desirable or predetermined environment. In addition, a pressure sensor 932 may be disposed within the vented volume 930 or in fluid communication with the vented volume 930 to determine the pressure within the vented volume 930. Such pressure measurements may be useful for determining the amount of fluid dispensed from the fluid reservoirs 917 or 919, determining when venting of the vented volume 930 is necessary, detecting leaks or clogs in the infusion pump system or the like.

In some cases, a drive mechanism 750 may be configured to expand or contract the first constrained variable volume 935 and second constrained variable volume 937 due to exertion of a translational force through a boundary section of the respective constrained variable volumes. In particular, when the drive mechanism 750 imparts axial force on the proximal section of the spool 902′, that axial force may be exerted against the contents of the constrained variable volumes 935′ and 937′ by the seals at distal end and proximal end of the volumes. The seal exerting force may depend on the direction of the axial force being exerted. As such, each of the seals 960, 962, 964 and 968 bounding the first and second constrained variable volumes 735′ and 737′ may form a boundary section of the constrained variable volume and axial force from the drive mechanism 750 may be applied to the contents of the constrained variable volume in this way. Resulting axial force may thus be applied by increased pressure on the contents of a constrained variable volume. If the constrained variable volume is in fluid communication with an inlet port or outlet port, the increased pressure or decreased pressure resulting from axial force being applied to the spool 902′ by the drive mechanism 750 may cause the constrained variable volume to expand or contract. Such an action may thus draw fluid into the expanding constrained variable volume or deliver fluid from a contracting constrained variable volume.

In some cases, the drive mechanism 750 and spool 902′ may be configured to translate the spool 902′ without the first constrained variable volume 935′ overlapping the second axial bore section 940′ or the second constrained variable volume 937′ overlapping the first axial bore section 935′. In some cases, the first axial bore section 901′ may include a plurality of first inlet ports 903′ in fluid communication with the first fluid reservoir 919 and the delivery mechanism 941 may be configured such that the first constrained variable volume 935′ is positionable to overlap all of the plurality of first inlet ports of the first fluid reservoir 919 independent of an overlap with an inlet port in fluid communication with another fluid reservoir. In some cases, the second axial bore section 940′ includes a plurality of second inlet ports 905′ in fluid communication with the second fluid reservoir 917 and the delivery mechanism 941 is configured such that the second constrained variable volume 937′ is positionable to overlap all of the plurality of second inlet ports 905′ of the second fluid reservoir 917 independent of an overlap with an inlet port in fluid communication with another fluid reservoir. Such a configuration is shown in the embodiment illustrated in FIG. 14A which may be applied to the embodiment of FIG. 17A. In some instances, the infusion pump may further include a proximal bore seal 961 and a distal bore seal 962 disposed between an outside surface of the spool 902′ and an inside surface 954 of the bore at respective proximal and distal ends of the spool 902′, the bore seals 961 and 962 being configured to isolate the inlet and outlet ports of the bore 900′ from the surrounding environment.

A delivery cycle for a delivery mechanism 941 embodiment such as shown in FIG. 17A may be initiated by using the drive mechanism 750 to advance the spool 902′ in a distal direction as indicated by the arrow 970 and disposing the second constrained variable volume 937′ in fluid communication with the second inlet port 905′ as shown in FIG. 17A. The second constrained variable volume 937′ may then be filled by further advancing the spool 902′ in a distal direction as indicated by the arrow 972 in FIG. 17B. The spool 902′ is then further advanced in a distal direction as shown in FIG. 17C until the second constrained variable volume 937′ is in fluid communication with the second outlet port 906′ as shown in FIG. 17D. Thereafter, axial force exerted on the spool 902′ by the drive mechanism 750 is reversed with the spool 902′ translating in a proximal direction as indicated by the arrow 974 in FIG. 17D. As the spool 902′ is translated in a proximal direction, the fourth seal 966 of the second constrained variable volume 937′ is axially fixed to the spool 902′ and thus moves closer to the third slidable seal 964 which is temporarily axially fixed in relation to the axial bore 900′ with the slide portion 953 of the spool 902′ sliding in sealed relation within an inner lumen 963 of the third seal 964. The approximation of the fourth seal 966 to the third seal 964 contracts the second constrained variable volume 937′ and thereby delivers the fluid disposed in the second volume from the second outlet port 906′ as shown by the arrow 976 in FIGS. 17D and 17E. The delivery of fluid from the second constrained variable volume 937′ continues until the third slidable seal 964 comes into contact with the distal stop 956 of the second volume 937′ as shown in FIG. 17F. Distal stop 956 is disposed opposite proximal stop 957 as shown in FIG. 17A. Fluid delivery ceases at this point and the third slidable seal 964 begins to move in a proximal direction along with the spool 902′.

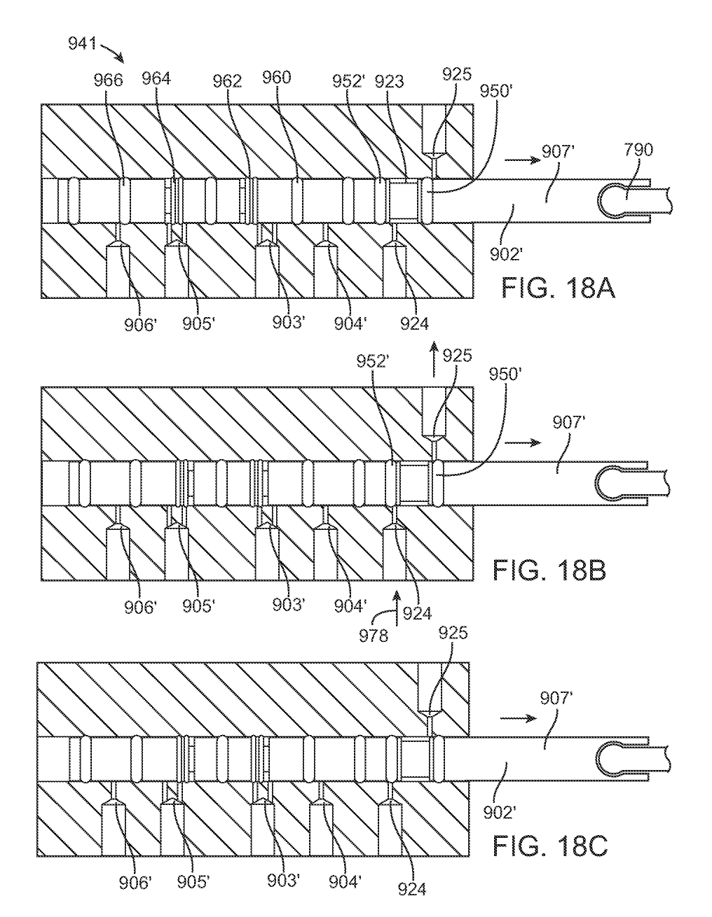

A vent cycle may be initiated by further translating the spool 902′ in a proximal direction as shown in FIG. 18A. The proximal translation of the spool 902′ is continued until the vent volume 923 of the spool 902′ disposed between a first vent volume seal 950′ and a second vent volume seal 952′ is in fluid communication with both the vent inlet port 924 and vent outlet port 925 as shown in FIG. 18B. In this position, a continuous fluid path is established between the vented volume 930 of the rigid shell 922 and the ambient atmosphere as shown by the arrows 978 in FIG. 18B. As discussed above, air or any other fluid disposed within the vented volume 930 may be vented to the atmosphere as shown by the arrows 978 in FIG. 18B. Air or any other fluid from the ambient atmosphere (or any other volume in fluid communication with the vent outlet port 925) may also be vented inward into the vented volume 930 in a direction opposite that of the arrows shown in FIG. 18B. Such a venting process would be typical as fluid is withdrawn from the first and second fluid reservoirs 917 and 919, air from the ambient atmosphere may be drawn into the vented volume 930 in order to fill the void left in the vented volume 930 by the dispensed fluid.

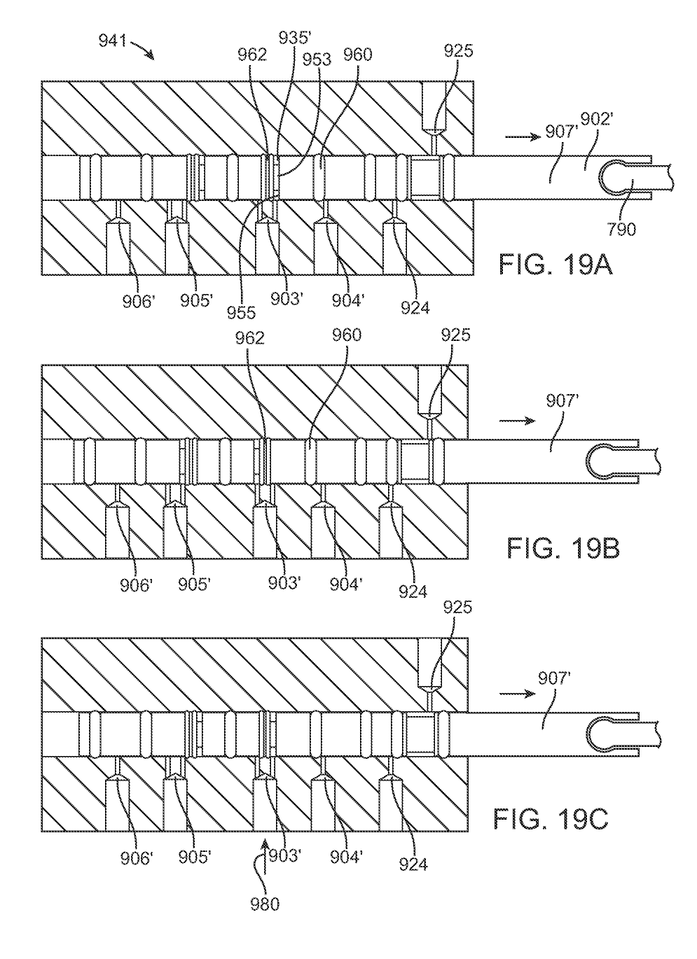

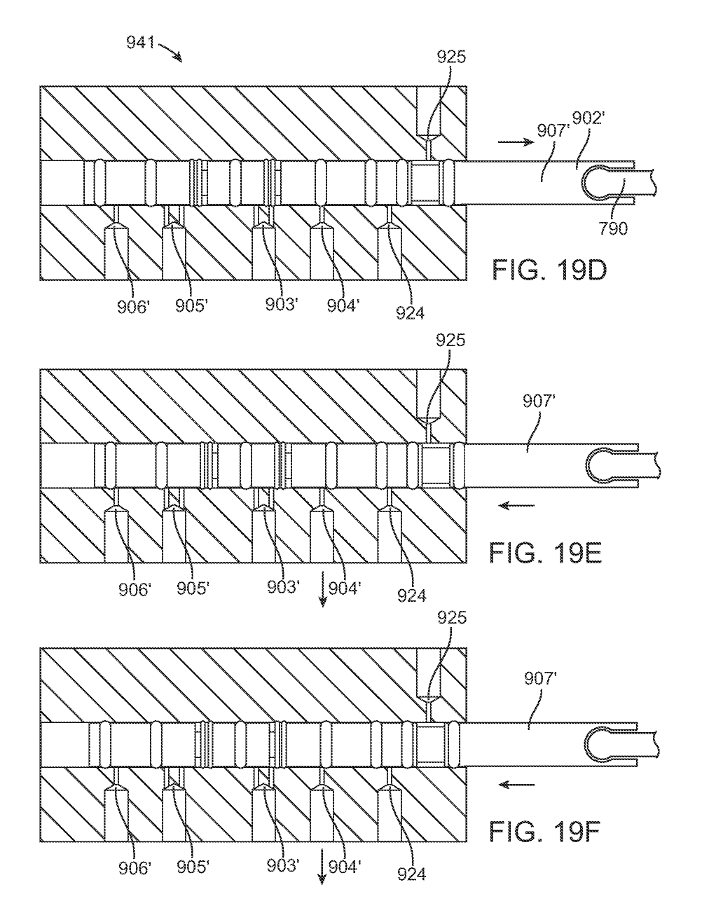

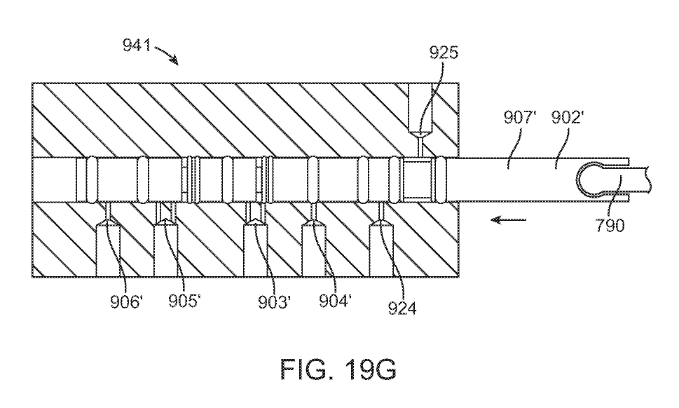

After venting, the spool 902′ may be further translated in a proximal direction in order to fill the first constrained variable volume 935′ as shown in FIG. 18C. FIG. 19B shows the first volume 935′ just prior to filling with fluid from the first fluid reservoir 919 through the first inlet port 903′. With the second slidable seal 962 disposed against the proximal stop 955 of the first slide portion 953 of the spool 902′, the spool 902′ is then axially translated in a proximal direction by the drive mechanism 750 as shown in FIG. 19C. As the spool 902′ moves proximally, the first constrained variable volume 935′ begins to fill as the separation between the first seal 960 and the second slidable seal 962 begins to increase and fluid is drawn from the first fluid reservoir 919 as indicated by the arrow 980 in FIG. 19C. The first seal 960 and second slidable seal 962 separate during proximal translation of the spool 902′ because the friction between the outer surface of the second seal 962 and inner surface 954 of the axial bore 900′ is greater than the friction force between the spool 902′ and the second seal 962. The filling continues until the first constrained variable volume 935′ is full and the second slidable seal 962 abuts the distal stop of the first slidable portion 953 of the spool 902′ as shown in FIG. 19C. At this point, the second seal 962 begins to move in a proximal axial direction with the spool 902′ to a position for delivery of the first volume 935′ as shown in FIG. 19D.

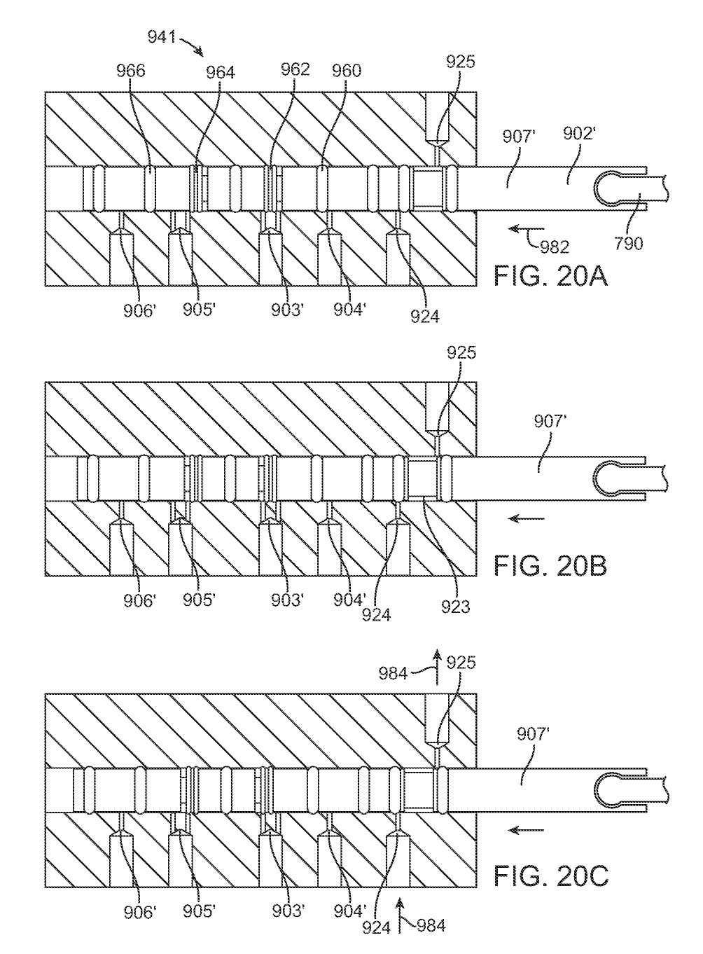

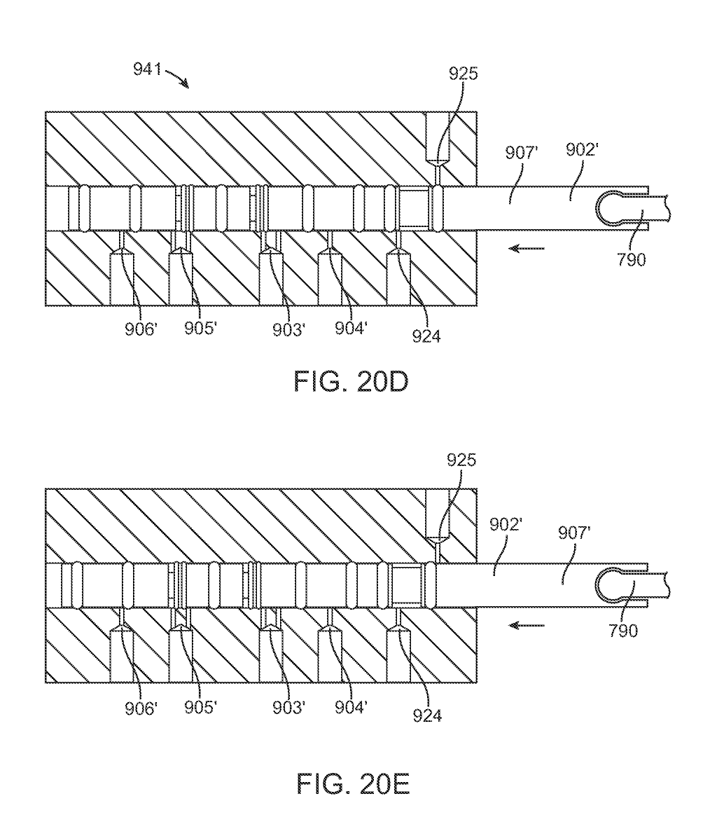

Once the filled first volume 935′ is in fluid communication with the first outlet port 904′, the direction of the spool movement may be reversed in order to initiate delivery of the fluid in the first volume 935′ through the first outlet port 904′ as shown in FIG. 19E. The spool 902′ may continue to be translated in a distal direction until the second slidable seal 962 contacts a proximal stop 955 of the first slide portion 953 of the spool 902′ and the first volume 935′ is at a minimum volume and delivery is complete as shown in FIG. 19F. The spool 902′ may then be returned to a start or home position as shown in FIG. 19G. After delivery of the fluid from the first constrained variable volume 935′ discussed above, the spool 902′ may be moved in a distal direction as shown by the arrow 982 in FIG. 20A towards a vent position. FIG. 20B shows the spool 902′ with the vent volume 923 of the spool 902′ positioned just prior to a venting arrangement. The vent volume 923 of the spool 902′ is shown in a vent position in FIG. 20C with the vent volume 923 of the spool 902′ in fluid communication with the vent inlet port 924 and vent outlet port 925 and venting taking place as indicated by arrows 984 of FIG. 20C. After venting, the spool 902′ may be translated back to a position for another delivery cycle for the second constrained variable volume 937′ as shown in FIG. 20D. FIG. 20E illustrates the spool 902 in a position just prior to filling of the second volume 937 as discussed above.

Though fluid was delivered from both the first fluid reservoir 919 and second fluid reservoir 917 for the delivery cycles discussed above, fluid may also be delivered from each of the fluid reservoirs 917 and 919 repeatedly and independently of delivery of fluid from the other fluid reservoir. For example, the second constrained variable volume 937′ may be repeatedly shuttled back and forth from a position in fluid communication with the second inlet port 905′ to a position in fluid communication with the second outlet port 906′ without ever making a fluid delivery to the first outlet port 904′. The second volume 937′ also never overlaps the first delivery section 901′ of the axial bore 900′ and vice versa. As such, there is no cross-contamination between the fluid within the first fluid reservoir 919 and the fluid within the second fluid reservoir 917. As discussed above, in some cases the first fluid reservoir 919 may contain a first therapeutic agent for delivery to a patient and the second fluid reservoir 917 may contain a second therapeutic agent for delivery to a patient. In addition, in some cases, the first therapeutic agent may include a fast acting insulin compound and the second therapeutic agent may include a slow acting insulin compound.

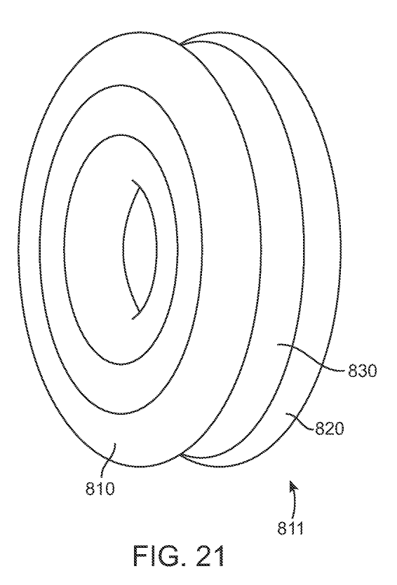

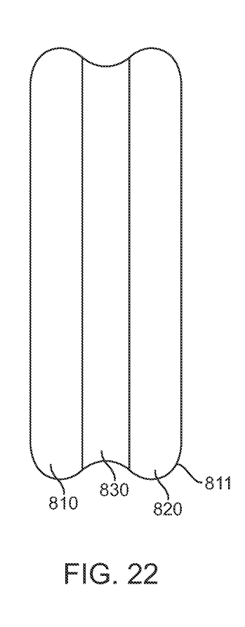

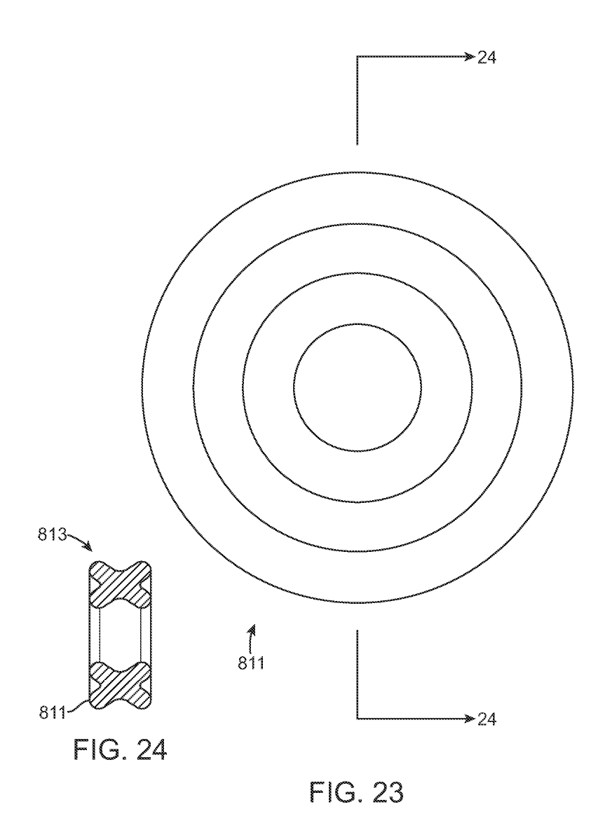

Referring to FIGS. 21-24, an annular o-ring type seal 811 is shown that may be particularly useful for any of the delivery mechanism embodiments shown herein that use annular type seals. FIG. 21 is a perspective view of the annular seal 811 with front and side views of the seal in FIGS. 22 and 23 respectively. The seal element may also include a first annular ring element 810 and a second annular ring element 820 disposed axially adjacent the first ring element with the first and second rings being conjoined or fused by a reduced thickness web 830 there between. FIG. 24 shows a transverse cross section of the seal element which has a substantially “X” shaped transverse cross section shape 813. The “X” shape 813 of the transverse cross section may be useful to prevent binding of the seal during axial displacement of the seal against a surface of the axial bore or slide portion of the spool. The cross section configuration also provides greater compliance with varying dimensions of inner tubular surfaces such as the inner surface of the axial bore and outer surfaces disposed within the inner lumen of seal, such as the slide portion of the spool of the embodiments shown in FIGS. 12A and 17A above.

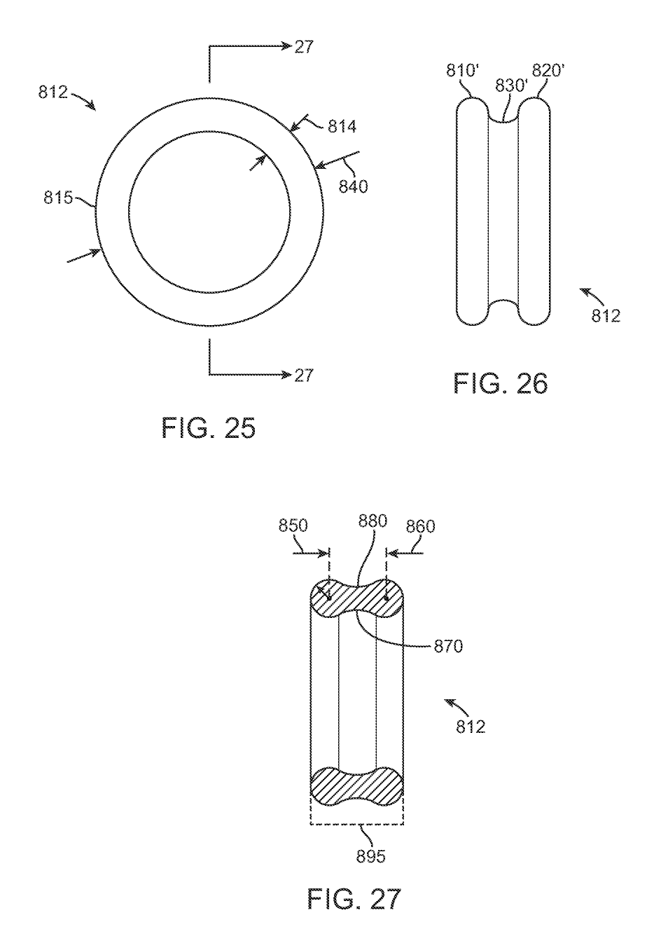

FIGS. 25-27 illustrate another embodiment of an annular seal embodiment 812 that may be useful for the delivery mechanism embodiments discussed herein. The twin style annular seal 812 shown in FIGS. 25-27 may be used in any of the suitable delivery mechanism embodiments discussed herein, such as for the spool seals of the spools of the delivery mechanisms. Some embodiments of the elastomeric annular seal 812 include an annular seal element 815 which has a substantially uniform cross section along a circumference thereof. The seal element 815 may also include a first annular ring element 810′ and a second annular ring element 820′ disposed axially adjacent the first ring element with the first and second rings being conjoined or fused by a reduced thickness web 830′ there between. The reduced thickness web is configured so as to form an inner annular channel 870 and an outer annular channel 880 between the first ring and second ring. In some cases, an axis 850 of the first ring element and an axis 860 of the second ring element may be separated by a distance equal to about 55 percent to about 70 percent of a transverse dimension 895 of the first and second ring elements 810′ or 820′. Some annular seal embodiments 812 may have an outer diameter 840 of about 2.0 mm to about 10.0 mm, more specifically, about 2.5 mm to about 3.0 mm. Some embodiments of the annular seal 812 may have a radial seal element thickness 814 of about 0.5 mm to about 1.0 mm. Some embodiments of the annular seal may have reduced thickness web 830′ of about 0.3 mm to about 0.7 mm. The material of the seal 812 may include any suitable material commonly used for o-rings such as silicone, nitrile rubber, fluoroelastomers, perfluoroelastomers, ethylene-propylene and the like.

With regard to the above detailed description, like reference numerals used therein may refer to like elements that may have the same or similar dimensions, materials and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” may refer to one of or a plurality of the elements it modifies (e.g., “a reagent” can mean one or more reagents) unless it is contextually clear either one of the elements or more than one of the elements is described. Although the present technology has been specifically disclosed by representative embodiments and optional features, modification and variation of the concepts herein disclosed may be made, and such modifications and variations may be considered within the scope of this technology.

Certain embodiments of the technology are set forth in the claim(s) that follow(s).

What is claimed is:

1. An infusion pump cartridge for delivering fluid to a patient, comprising:
a delivery mechanism including:
a first inlet port,
a second inlet port spaced from the first inlet port,
at least one outlet port which is spaced from the first inlet port and second inlet port, and
a constrained variable volume which is translatable between a position in fluid communication with the first inlet port, a position in fluid communication with the second inlet port and a position in fluid communication with an outlet port and which is configured to expand while in fluid communication with an inlet port and contract while in fluid communication with an outlet port;
a first fluid reservoir including an interior volume in fluid communication with the first inlet port; and
a second fluid reservoir including an interior volume in fluid communication with the second inlet port.

2. The cartridge of claim 1 wherein the constrained variable volume is configured to expand and contract due to exertion of a translational force on a boundary section of the constrained variable volume.

3. An infusion pump for delivering fluids to a patient, comprising:
a delivery mechanism including:
a first inlet port,
a second inlet port spaced from the first inlet port,
at least one outlet port which is spaced from the first inlet port and second inlet port, and
a constrained variable volume translatable between a position in fluid communication with the first inlet port, a position in fluid communication with the second inlet port and a position in fluid communication with an outlet port;
a first fluid reservoir including an interior volume in fluid communication with the first inlet port;
a second fluid reservoir including an interior volume in fluid communication with the second inlet port; and
a drive mechanism which is operatively coupled to the constrained variable volume, which is configured to translate the constrained variable volume from each of the inlet ports to an outlet port and which is configured to expand and contract the constrained variable volume of the spool.

4. The infusion pump of claim 1 wherein the drive mechanism is configured to expand or contract the constrained variable volume by exerting translational force through a boundary section of the constrained variable volume.

5. An infusion pump for delivering fluids to a patient, comprising:
a delivery mechanism including:
an axial bore including a longitudinal axis and an interior volume,
a first inlet port in fluid communication with the interior volume of the axial bore,
a second inlet port axially spaced from the first inlet port and in fluid communication with the interior volume of the axial bore,
at least one outlet port which is axially spaced from the first inlet port and second inlet port and which is in fluid communication with the interior volume of the axial bore, and
a spool which is disposed within the axial bore, which is axially translatable within the axial bore and which forms a constrained variable volume in conjunction with an interior surface of the axial bore;
a first fluid reservoir including an interior volume in fluid communication with the first inlet port;
a second fluid reservoir including an interior volume in fluid communication with the second inlet port; and
a drive mechanism which is operatively coupled to the spool, which is configured to axially translate the constrained variable volume from each of the inlet ports to an outlet port and which is configured to expand or contract the constrained variable volume of the spool by exerting translational axial force through a boundary section of the constrained variable volume.

6. The infusion pump of claim 5 wherein the drive mechanism is configured to expand or contract the constrained variable volume by exerting translational axial force through a boundary section of the constrained variable volume.

7. The infusion pump of claim 5 wherein the axial bore, first inlet port, second inlet port and at least one outlet port are disposed within a housing of the delivery mechanism.

8. The infusion pump of claim 5 wherein the bore comprises a plurality of inlet ports in fluid communication with a single fluid reservoir and the delivery mechanism is configured such that the constrained variable volume is positionable to overlap all of the plurality of inlet ports of this single fluid reservoir independent of an overlap with an inlet port in fluid communication with another fluid reservoir.

9. The infusion pump of claim 5 wherein the constrained variable volume is formed between a first seal disposed around the spool, a second seal disposed around the spool, an outer surface of the spool body at least between the first and second seal and an interior surface of the bore between the first and second seal, the first and second seals being axially translatable relative to each other but mechanically constrained to a maximum and minimum axial separation.

10. The infusion pump of claim 9 wherein the first seal is disposed around a first section of the spool and the second seal is disposed around a second section of the spool and wherein the first section of the spool and second section of the spool are coupled together by a limited displacement coupling which is configured to allow axial movement of the first and second spool sections relative to each other for a limited axial distance.

11. The infusion pump of claim 10 wherein the drive mechanism is coupled to the first spool section and coupled to the second spool section through the limited displacement coupling.

12. The infusion pump of claim 9 wherein the variable volume of the spool is bounded by an outside surface of the spool, an inside surface of the bore, a first seal disposed between the spool and the bore, the first seal being substantially axially fixed relative to the spool but displaceable relative to an inside surface of the bore and a second seal which is disposed between an outside surface of a slide portion of the spool and inside surface of the bore, which is configured to slide over the slide portion of the spool, which forms a substantially fluid tight but displaceable seal between an outside surface of the slide portion and the inside surface of the bore and which has a greater frictional resistance to sliding against the inside surface of the bore relative to the frictional resistance to sliding against the slide portion of the spool.

13. The infusion pump of claim 12 wherein a shaft of the spool is axially continuous and rigid along an axial direction and the drive mechanism is directly coupled to the rigid shaft.

14. The infusion pump of claim 12 wherein the slide portion of the spool comprises a smooth reduced diameter axial section of a shaft of the spool having a substantially continuous transverse cross section, a first stop at an end of the slide portion which is configured to limit axial movement of the second seal over the slide portion and a second stop opposite the slide portion of the first stop that is also configured to limit axial movement of the second seal over the slide portion.

15. The infusion pump of claim 5 wherein an axial separation of the first inlet port from the second inlet port is greater than a maximum axial length of the constrained variable volume.

16. The infusion pump of claim 5 wherein an axial separation of the inlet ports from the outlet port is greater than a maximum axial length of the constrained variable volume.

17. The infusion pump of claim 5 wherein an axial separation of the first inlet port from the second inlet port is greater than a maximum displacement between the first seal and the second seal.

18. The infusion pump of claim 5 wherein a minimum axial separation of the inlet ports from the outlet port is greater than a maximum displacement between the first seal and the second seal.

19. The infusion pump of claim 5 further comprising a proximal bore seal and a distal bore seal disposed between an outside surface of the spool and the inside surface of the bore at respective proximal and distal ends of the spool, the bore seals being configured to isolate the inlet and outlet ports of the bore from the surrounding environment.

20. The infusion pump of claim 5 wherein the first and second fluid reservoirs comprise collapsible reservoirs bounded by a thin flexible fluid tight material.

21. The infusion pump of claim 20 further comprising a substantially rigid fluid tight shell disposed about the first and second fluid reservoirs with a fluid tight interior volume being formed between an inside surface of the rigid shell and respective outside surfaces of the first and second fluid reservoirs.

22. The infusion pump of claim 21 wherein the interior volume of the rigid shell comprises a vented volume and further comprising a vent port in fluid communication between the interior volume of the bore and an interior volume of the vented volume and a vent outlet port in fluid communication between an interior volume of the bore and the ambient atmosphere.

23. The infusion pump of claim 22 further comprising a pressure sensor disposed in operative fluid communication with the vented volume.

24. The infusion pump of claim 5 further comprising a controller operatively coupled to the drive mechanism.

25. The infusion pump of claim 24 wherein the controller comprises at least one processor and a memory device operatively coupled to the processor.

26. The infusion pump of claim 24 further comprising a graphic user interface operatively coupled to the controller.

27. The infusion pump of claim 26 wherein the graphic user interface comprises a touch sensitive screen.

28. The infusion pump of claim 5 wherein the first fluid reservoir contains a first therapeutic agent for delivery to a patient and the second fluid reservoir contains a second therapeutic agent for delivery to a patient.

29. The infusion pump of claim 28 wherein the first therapeutic agent comprises a fast acting insulin compound and the second therapeutic agent comprises a slow acting insulin compound.

30. The infusion pump of claim 5 wherein the delivery mechanism comprises a plurality of outlet ports and wherein the drive mechanism is configured to impart controlled axial movement on the spool and translate the variable volume of the spool from each of the inlet ports to each of the plurality of outlet ports.

31. The infusion pump of claim 5 wherein the drive mechanism is coupled to the spool with a ball and socket coupling.

32. The infusion pump of claim 5 wherein the drive mechanism comprises a rack and pinion configuration with and end of the rack being coupled to the spool of the delivery mechanism.

33. The infusion pump of claim 5 further comprising a fluid conduit including an inner lumen with a first end of the inner lumen in fluid communication with a patient's body and a second end of the inner lumen in fluid communication with the outlet port of the delivery mechanism.

34. An infusion pump for delivering fluids to a patient, comprising:
a disposable component comprising:
a delivery mechanism including:
an axial bore, a first inlet port in fluid communication with an interior volume of the axial bore, a second inlet port axially spaced from the first inlet port and in fluid communication with the interior volume of the axial bore, and at least one outlet port which is axially spaced from the inlet ports and which is in fluid communication with the interior volume of the axial bore, and
a spool which is disposed within the axial bore, which is axially translatable within the axial bore, and which forms a constrained variable volume in conjunction with an interior surface of the axial bore;
a first fluid reservoir including an interior volume in fluid communication with the first inlet port;
a second fluid reservoir including an interior volume in fluid communication with the second inlet port; and
a reusable component comprising a drive mechanism which is operatively coupled to the spool, which is configured to impart controlled axial movement on the spool and translate the constrained variable volume from each of the inlet ports to the at least one outlet port and which is configured to expand or contract the constrained variable volume of the spool by exerting translational axial force through a boundary section of the constrained variable volume.

35. The infusion pump of claim 34 wherein the drive mechanism is detachably coupled to the spool.

36. The infusion pump of claim 34 wherein the reusable component further comprises a controller operatively coupled to the drive mechanism.

37. The infusion pump of claim 34 wherein the reusable component further comprises a power storage cell operatively coupled to the drive mechanism.

38. The infusion pump of claim 37 wherein the power storage cell comprises a battery.

39. The infusion pump of claim 34 wherein the first fluid reservoir contains a first therapeutic agent for delivery to a patient and the second fluid reservoir contains a second therapeutic agent different from the first therapeutic agent for delivery to a patient.

40. The infusion pump of claim 39 wherein the first therapeutic agent comprises insulin.

41. The infusion pump of claim 40 wherein the first therapeutic agent comprises a first type of insulin and the second therapeutic agent comprises a second type of insulin different from the first type.

42. A method of delivering fluid to a patient from two independent fluid reservoirs, comprising:
providing an infusion pump, comprising:
a delivery mechanism including:
a first inlet port,
a second inlet port spaced from the first inlet port,
at least one outlet port which is spaced from the first inlet port and second inlet port, and
a constrained variable volume translatable between a position in fluid communication with the first inlet port, a position in fluid communication with the second inlet port and a position in fluid communication with an outlet port;
a first fluid reservoir including an interior volume in fluid communication with the first inlet port;
a second fluid reservoir including an interior volume in fluid communication with the second inlet port; and
a drive mechanism which is operatively coupled to the constrained variable volume, which is configured to axially translate the constrained variable volume from each of the inlet ports to an outlet port and which is configured to expand or contract the constrained variable volume of the spool;
initiating a dispense cycle by translating the constrained variable volume into a position in fluid communication with the first inlet port;
exerting translational force through a boundary section of the constrained variable volume to expand the constrained variable volume and draw fluid into the constrained variable through the first inlet port from the first reservoir;
translating the constrained variable volume into a position in fluid communication with an outlet port;
exerting translational force through a boundary section of the constrained variable volume so as to at least partially contract the constrained variable volume and dispense fluid from the constrained variable volume through the outlet port to a patient;
translating the constrained variable volume to a position in fluid communication with the second inlet port;
exerting translational force through a boundary section of the constrained variable volume to expand the constrained variable volume and draw fluid into the constrained variable through the second inlet port from the second reservoir;
translating the constrained variable volume to a position in fluid communication with an outlet port; and
exerting translational force through a boundary section of the constrained variable volume so as to at least partially contract the constrained variable volume and dispense fluid from the constrained variable volume through the outlet port to a patient.

43. The method of claim 42 wherein the first fluid reservoir contains a first therapeutic agent for delivery to a patient and the second fluid reservoir contains a second therapeutic agent different from the first therapeutic agent for delivery to a patient and further comprising delivering a first therapeutic agent and a second therapeutic agent different from the first therapeutic agent to a patient.

44. The method of claim 42 wherein the infusion pump comprises at least three inlet ports and respective fluid reservoirs and further comprising:
translating the constrained variable volume to a position in fluid communication with a third inlet port;
exerting translational force through a boundary section of the constrained variable volume to expand the constrained variable volume and draw fluid into the constrained variable through the third inlet port from a third reservoir;
translating the constrained variable volume to a position in fluid communication with an outlet port; and
exerting translational force through a boundary section of the constrained variable volume so as to at least partially contract the constrained variable volume and dispense fluid from the constrained variable volume through the outlet port to a patient.

45. An infusion pump cartridge for delivering fluid to a patient, comprising:
a delivery mechanism including:
a first delivery section which includes a first inlet port and a first outlet port,
a first constrained variable volume which is translatable between a position in fluid communication with the first inlet port and a position in fluid communication with the first outlet port and which is configured to expand while in fluid communication with the first inlet port and contract while in fluid communication with the first outlet port,
a second delivery section which includes a second inlet port and a second outlet port, and
a second constrained variable volume which is coupled to the first constrained variable volume, which is translatable between a position in fluid communication with the second inlet port and a position in fluid communication with the second outlet port and which is configured to expand while in fluid communication with the second inlet port and contract while in fluid communication with the second outlet port;
a first fluid reservoir including an interior volume in fluid communication with the first inlet port; and
a second fluid reservoir including an interior volume in fluid communication with the second inlet port.

46. The infusion pump cartridge of claim 45 wherein the first constrained variable volume is configured to expand or contract due to exertion of a translational force through a boundary section of the first constrained variable volume and the second constrained variable volume is configured to expand or contract due to exertion of a translational force through a boundary section of the second constrained variable volume.

47. An infusion pump, comprising:
a delivery mechanism including:
a first delivery section which includes a first inlet port and a first outlet port,
a first constrained variable volume which is translatable between a position in fluid communication with the first inlet port and a position in fluid communication with the first outlet port,
a second delivery section which includes a second inlet port and a second outlet port, and
a second constrained variable volume which is translatable between a position in fluid communication with the second inlet port and a position in fluid communication with the second outlet port;
a first fluid reservoir including an interior volume in fluid communication with the first inlet port;
a second fluid reservoir including an interior volume in fluid communication with the second inlet port; and
a drive mechanism which is operatively coupled to the first constrained variable volume and operatively coupled to the second constrained variable volume.

48. The infusion pump of claim 47 wherein the drive mechanism is:
configured to translate the first constrained variable volume between the first inlet port and first outlet port,
configured to expand the first constrained variable volume while in fluid communication with the first inlet port and contract the first constrained variable volume while in fluid communication with the first outlet,
configured to translate the second constrained variable volume between the second inlet port and second outlet port, and
configured to expand the second constrained variable volume while in fluid communication with the second inlet port and contract the second constrained variable volume while in fluid communication with the second outlet port.

49. The infusion pump of claim 48 wherein the drive mechanism is configured to expand or contract the first constrained variable volume by exerting translational force through a boundary section of the first constrained variable volume and configured to expand or contract the second constrained variable volume by exerting translational force through a boundary section of the second constrained variable volume.

50. An infusion pump for delivering fluids to a patient, comprising:
a disposable component comprising:
a delivery mechanism including:
a first delivery section which includes a first inlet port and a first outlet port,
a first constrained variable volume which is translatable between a position in fluid communication with the first inlet port and a position in fluid communication with the first outlet port,
a second delivery section which includes a second inlet port and a second outlet port, and
a second constrained variable volume which is translatable between a position in fluid communication with the second inlet port and a position in fluid communication with the second outlet port;
a first fluid reservoir including an interior volume in fluid communication with the first inlet port;
a second fluid reservoir including an interior volume in fluid communication with the second inlet port; and
a reusable component comprising a drive mechanism which is operatively coupled to the first constrained variable volume and operatively coupled to the second constrained variable volume.

51. The infusion pump of claim 50 wherein the reusable component further comprises a controller operatively coupled to the drive mechanism.

52. The infusion pump of claim 50 wherein the reusable component further comprises a power storage cell operatively coupled to the drive mechanism.

53. The infusion pump of claim 52 wherein the power storage cell comprises a battery.

54. The infusion pump of claim 50 wherein the first fluid reservoir contains a first therapeutic agent for delivery to a patient and the second fluid reservoir contains a second therapeutic agent different from the first therapeutic agent for delivery to a patient.

55. The infusion pump of claim 54 wherein the first therapeutic agent comprises insulin.

56. The infusion pump of claim 55 wherein the first therapeutic agent comprises a first type of insulin and the second therapeutic agent comprises a second type of insulin different from the first type.

57. An infusion pump, comprising:
a delivery mechanism including:
a first axial bore section which includes a first inlet port and a first outlet port in fluid communication with an interior volume of the first axial bore section,
a first constrained variable volume with a maximum axial length less than a distance between the first inlet port and first outlet port,
a second axial bore section which includes a second inlet port and second outlet port in fluid communication with an interior volume of the second axial bore section, and
a second constrained variable volume with a maximum axial length less than a distance between the second inlet port and second outlet port;
a first fluid reservoir including an interior volume in fluid communication with the first inlet port;
a second fluid reservoir including an interior volume in fluid communication with the second inlet port; and
a drive mechanism which is operatively coupled to the first constrained variable volume and which is operatively coupled to the second constrained variable volume.

58. The infusion pump of claim 57 wherein the drive mechanism is:
configured to axially translate the first variable volume between the first inlet port and first outlet port,
configured to expand the first constrained variable volume while in fluid communication with the first inlet port and contract the first constrained variable volume while in fluid communication with the first outlet port,
configured to axially translate the second variable volume between the second inlet port and second outlet port, and
configured to expand the second constrained variable volume while in fluid communication with the second inlet port and contract the second constrained variable volume while in fluid communication with the second outlet port.

59. The infusion pump of claim 58 wherein the drive mechanism is configured to expand or contract the first variable volume by exerting translational axial force through a boundary section of the first variable volume and configured to expand or contract the second variable volume by exerting translational axial force through a boundary section of the second variable volume.

60. An infusion pump, comprising:
a delivery mechanism including:
an axial bore including:
a first axial bore section which includes a first inlet port and a first outlet port with said ports being in fluid communication with an interior volume of the first axial bore section,
a second axial bore section which includes a second inlet port and second outlet port with said ports being in fluid communication with an interior volume of the second axial bore section, and
a spool which is disposed within the axial bore, which is axially translatable within the axial bore, and which comprises:
a first spool section including a proximal end configured to couple to a drive mechanism and a first seal which forms a fluid tight seal between the first spool section and an interior surface of the axial bore and which is axially fixed relative to the first spool section and slidable relative to the interior surface of the axial bore,
a second spool section including a proximal end coupled to a distal end of the first spool section by a limited displacement coupling and a second seal which forms a fluid tight seal between the second spool section and the interior surface of the axial bore and which is axially fixed relative to the second spool section and slidable relative to the interior surface of the axial bore,
a third spool section including a proximal end coupled to a distal end of the second spool section by a limited displacement coupling and a third seal which forms a fluid tight seal between the third spool section and the interior surface of the axial bore and which is axially fixed relative to the third spool section and axially slidable relative to the interior surface of the axial bore,
a first constrained variable volume formed between the first spool section, the second spool section, the first seal, the second seal and the interior surface of the axial bore;
a second constrained variable volume formed between the second spool section, the third spool section, the second seal, the third seal and the interior surface of the axial bore;
a first fluid reservoir including an interior volume in fluid communication with the first inlet port;
a second fluid reservoir including an interior volume in fluid communication with the second inlet port; and
a drive mechanism which is operatively coupled to the proximal end of the spool.

61. The infusion pump of claim 60 wherein the drive mechanism is:
configured to axially translate the first constrained variable volume between the first inlet port and first outlet port,
configured to expand the first constrained variable volume while in fluid communication with the first inlet port and contract the first constrained variable volume while in fluid communication with the first outlet port,
configured to axially translate the second variable volume between the second inlet port and second outlet port, and
configured to expand the second constrained variable volume while in fluid communication with the second inlet port and contract the second constrained variable volume while in fluid communication with the second outlet port.

62. The infusion pump of claim 61 wherein the drive mechanism is configured to expand or contract the first constrained variable volume by exerting translational axial force through a boundary section of the first constrained variable volume and configured to expand or contract the second constrained variable volume by exerting translational axial force through a boundary section of the second constrained variable volume.

63. The infusion pump of claim 60 wherein the first and second seals are axially translatable relative to each other but mechanically constrained to a maximum and minimum axial separation over a limited axial distance by the limited displacement coupling operatively coupled between the first spool section and second spool section.

64. The infusion pump of claim 60 wherein the second and third seals are axially translatable relative to each other but mechanically constrained to a maximum and minimum axial separation over a limited axial distance by the limited displacement coupling operatively coupled between the second spool section and third spool section.

65. The infusion pump of claim 60 wherein the drive mechanism and spool are configured to translate the spool without the first constrained variable volume overlapping the second axial bore section or the second constrained variable volume overlapping the first axial bore section.

66. The infusion pump of claim 60 wherein the first axial bore section comprises a plurality of first inlet ports in fluid communication with the first fluid reservoir and the delivery mechanism is configured such that the first constrained variable volume is positionable to overlap all of the plurality of first inlet ports of the first fluid reservoir independent of an overlap with an inlet port in fluid communication with another fluid reservoir.

67. The infusion pump of claim 60 wherein the second axial bore section comprises a plurality of second inlet ports in fluid communication with the second fluid reservoir and the delivery mechanism is configured such that the second constrained variable volume is positionable to overlap all of the plurality of second inlet ports of the second fluid reservoir independent of an overlap with an inlet port in fluid communication with another fluid reservoir.

68. The infusion pump of claim 60 further comprising a proximal bore seal and a distal bore seal disposed between an outside surface of the spool and an inside surface of the bore at respective proximal and distal ends of the spool, the bore seals being configured to isolate the inlet and outlet ports of the bore from the surrounding environment.

69. The infusion pump of claim 60 wherein the first and second fluid reservoirs comprise collapsible reservoirs bounded by a thin flexible fluid tight material.

70. The infusion pump of claim 69 further comprising a substantially rigid fluid tight shell disposed about the first and second fluid reservoirs with a fluid tight interior volume being formed between an inside surface of the rigid shell and respective outside surfaces of the first and second fluid reservoirs.

71. The infusion pump of claim 70 wherein the interior volume of the rigid shell comprises a vented volume and further comprising a vent port in fluid communication between the interior volume of the bore and an interior volume of the vented volume and a vent outlet port in fluid communication between an interior volume of the bore and the ambient atmosphere.

72. The infusion pump of claim 71 further comprising a pressure sensor disposed in operative fluid communication with the vented volume.

73. The infusion pump of claim 60 further comprising a controller operatively coupled to the drive mechanism.

74. The infusion pump of claim 73 wherein the controller comprises at least one processor and a memory device operatively coupled to the processor.

75. The infusion pump of claim 73 further comprising a graphic user interface operatively coupled to the controller.

76. The infusion pump of claim 75 wherein the graphic user interface comprises a touch sensitive screen.

77. The infusion pump of claim 60 wherein the first fluid reservoir contains a first therapeutic agent for delivery to a patient and the second fluid reservoir contains a second therapeutic agent for delivery to a patient.

78. The infusion pump of claim 77 wherein the first therapeutic agent comprises a fast acting insulin compound and the second therapeutic agent comprises a slow acting insulin compound.

79. An infusion pump, comprising:
a delivery mechanism including:
an axial bore including:
a first axial bore section which includes a first inlet port and a first outlet port with said first inlet and first outlet ports being in fluid communication with an interior volume of the first axial bore section,
a second axial bore section which is axially spaced from the first axial bore section and which includes a second inlet port and second outlet port with said second inlet and outlet ports being in fluid communication with an interior volume of the second axial bore section, and
a spool which includes a proximal end and a distal end, which is disposed within the axial bore, which is axially translatable within the axial bore, which forms a first constrained variable volume between a first seal axially fixed relative to the spool, a slidable second seal disposed distally of the first seal, and an interior surface of the axial bore and which forms a second constrained variable volume between a third slidable seal disposed distally of the second slidable seal, a fourth seal disposed distally of the third slidable seal and axially fixed relative to the spool and an interior surface of the axial bore;
a first fluid reservoir including an interior volume in fluid communication with the first inlet port;
a second fluid reservoir including an interior volume in fluid communication with the second inlet port; and
a drive mechanism which is operatively coupled to the proximal end of the spool.

80. The infusion pump of claim 79 wherein the second slidable seal and third slidable seal are configured to slide over a slide portion of the spool, the slidable seals forming a substantially fluid tight but displaceable seal between an outside surface of a respective slide portion of the spool and the inside surface of the bore and configured to have a greater frictional resistance to sliding against the inside surface of the bore relative to the frictional resistance to sliding against the slide portion of the spool.

81. The infusion pump of claim 80 wherein the slide portions of the spool comprise smooth reduced diameter axial sections of a shaft of the spool, the reduced diameter sections having a substantially continuous transverse cross section, a first stop at an end of the slide portion which is configured to limit axial movement of the slidable seals over the slide portion and a second stop opposite the first stop that is also configured to limit axial movement of the slidable seals over the slide portion and define relative constrained variable volumes.

82. The infusion pump of claim 79 wherein a shaft of the spool is axially continuous and rigid along an axial direction and the drive mechanism is directly coupled to the proximal end of the rigid shaft.

83. The infusion pump of claim 79 wherein the drive mechanism is:
configured to axially translate the first constrained variable volume between the first inlet port and first outlet port,
configured to expand the first constrained variable volume while in fluid communication with the first inlet port and contract the first constrained variable volume while in fluid communication with the first outlet port,
configured to axially translate the second variable volume between the second inlet port and second outlet port, and
configured to expand the second constrained variable volume while in fluid communication with the second inlet port and contract the second constrained variable volume while in fluid communication with the second outlet port.

84. The infusion pump of claim 80 wherein the drive mechanism is configured to expand or contract the first constrained variable volume by exerting translational axial force through a boundary section of the first constrained variable volume and configured to expand or contract the second constrained variable volume by exerting translational axial force through a boundary section of the second constrained variable volume.

85. The infusion pump of claim 81 wherein the first and second seals are axially translatable relative to each other but mechanically constrained to a maximum and minimum axial separation over a limited axial distance by the stops that confine the second seal.

86. The infusion pump of claim 81 wherein the third and fourth seals are axially translatable relative to each other but mechanically constrained to a maximum and minimum axial separation over a limited axial distance by the stops that confine the third seal.

87. The infusion pump of claim 79 wherein the drive mechanism and spool are configured to translate the spool without the first constrained variable volume overlapping the second axial bore section or the second constrained variable volume overlapping the first axial bore section.

88. The infusion pump of claim 79 wherein the first axial bore section comprises a plurality of first inlet ports in fluid communication with the first fluid reservoir and the delivery mechanism is configured such that the first constrained variable volume is positionable to overlap all of the plurality of first inlet ports of the first fluid reservoir independent of an overlap with an inlet port in fluid communication with another fluid reservoir.

89. The infusion pump of claim 79 wherein the second axial bore section comprises a plurality of second inlet ports in fluid communication with the second fluid reservoir and the delivery mechanism is configured such that the second constrained variable volume is positionable to overlap all of the plurality of second inlet ports of the second fluid reservoir independent of an overlap with an inlet port in fluid communication with another fluid reservoir.

90. The infusion pump of claim 79 further comprising a proximal bore seal and a distal bore seal disposed between an outside surface of the spool and an inside surface of the bore at respective proximal and distal ends of the spool, the bore seals being configured to isolate the inlet and outlet ports of the bore from the surrounding environment.

91. The infusion pump of claim 79 wherein the first and second fluid reservoirs comprise collapsible reservoirs bounded by a thin flexible fluid tight material.

92. The infusion pump of claim 91 further comprising a substantially rigid fluid tight shell disposed about the first and second fluid reservoirs with a fluid tight interior volume being formed between an inside surface of the rigid shell and respective outside surfaces of the first and second fluid reservoirs.

93. The infusion pump of claim 92 wherein the interior volume of the rigid shell comprises a vented volume and further comprising a vent port in fluid communication between the interior volume of the bore and an interior volume of the vented volume and a vent outlet port in fluid communication between an interior volume of the bore and the ambient atmosphere.

94. The infusion pump of claim 93 further comprising a pressure sensor disposed in operative fluid communication with the vented volume.

95. The infusion pump of claim 79 further comprising a controller operatively coupled to the drive mechanism.

96. The infusion pump of claim 95 wherein the controller comprises at least one processor and a memory device operatively coupled to the processor.

97. The infusion pump of claim 95 further comprising a graphic user interface operatively coupled to the controller.

98. The infusion pump of claim 97 wherein the graphic user interface comprises a touch sensitive screen.

99. The infusion pump of claim 79 wherein the first fluid reservoir contains a first therapeutic agent for delivery to a patient and the second fluid reservoir contains a second therapeutic agent for delivery to a patient.

100. The infusion pump of claim 99 wherein the first therapeutic agent comprises a fast acting insulin compound and the second therapeutic agent comprises a slow acting insulin compound.

101. An elastomeric annular seal, comprising an annular seal element which includes a substantially uniform cross section along a circumference thereof, the seal element including a first annular ring element and a second annular ring disposed axially adjacent the first ring with the first and second rings being conjoined or fused by a reduced thickness web therebetween so as to form an inner annular channel and an outer annular channel between the rings.

102. The annular seal of claim 101 wherein an axis of the first ring element and an axis of the second ring element are separated by a distance equal to about 55 percent to about 70 percent of a transverse dimension of the first and second ring elements.

103. The annular seal of claim 101 wherein the seal is molded in a continuous monolithic structure from a single material.

104. The annular seal of claim 103 wherein the seal comprises a polymer.

105. The annular seal of claim 104 wherein the seal comprises Nitrile® polymer.

106. The annular seal of claim 101 wherein the seal element comprises a polymer including a shore hardness of about 65 shore A to about 75 shore A.

107. The annular seal of claim 101 wherein the seal has an outer diameter of about 2.0 mm to about 10.0 mm.

108. The annular seal of claim 101 wherein the seal element comprises a transverse thickness of about 0.5 mm to about 1.0 mm.
